(19) United States
(12) Patent Application Publication
(10) Pub. No.: US 2009/0136986 A1
(43) Pub. Date: May 28, 2009

(54) METHODS AND CELLS FOR CREATING FUNCTIONAL DIVERSITY AND USES THEREOF

(75) Inventors: George Church, Brookline, MA (US); Brian M. Baynes, Cambridge, MA (US)

Correspondence Address: WOLF GREENFIELD & SACKS, P.C., 600 ATLANTIC AVENUE, BOSTON, MA, 02210-2206, US

(73) Assignee: Codon Devices, Inc., Cambridge, MA (US)

(21) Appl. No.: 12/273,098

(22) Filed: Nov. 18, 2008

Related U.S. Application Data
(63) Continuation of application No. PCT/US2007/012077, filed on May 19, 2007.
(60) Provisional application No. 60/801,833, filed on May 19, 2006.

(51) Int. Cl. C12Q 1/02; C12N 15/74

(52) U.S. Cl. 435/29; 435/471

(57) ABSTRACT

The invention provides methods and compositions relating to cells having altered functions, and the nucleic acids that impart those functions. Altered cellular function arises from in vivo directed recombination of genetic elements to yield a recombined nucleic acid. These methods and compositions may utilize altered host cells having altered recombination enzyme profiles and/or altered recombination sites. The invention involves in some aspects methods for assembling nucleic acid molecules, such as genomic DNA. Aspects of the invention also provide kits, compositions, devices, and systems for generating novel recombined nucleic acids and cells having altered cell function.

### RELATED APPLICATIONS

This application claims the benefit under 35 U.S.C. 119(e) from U.S. provisional application Ser. No. 60/801,833, filed May 19, 2006, the entire contents of which are herein incorporated by reference.

### FIELD OF THE INVENTION

This invention pertains to the field of genome synthesis and directed evolution. The invention provides methods for improving cellular functions.

### BACKGROUND

Cellular and organism genomes are naturally mutated by a variety of mechanisms and as a result of exposure to various environments. Natural mutation however is generally random and thus not particularly efficient. Random nucleic acid mutation has also been attempted in controlled laboratory settings. However, it too is not very efficient and requires screening of vast numbers of mutants to identify a candidate of interest.

Methods for mutating cells or organisms in a more ordered and directed manner would facilitate the identification of mutants of interest.

### SUMMARY OF THE INVENTION

Aspects of the invention relate to nucleic acid libraries and host cells that can be used to generate a variety of different functional nucleic acid configurations in vivo. Certain aspects of the invention involve identifying genetic configurations that provide one or more biological functions of interest. In some embodiments, new or alternative regulatory or metabolic pathways may be identified. In some embodiments, methods of producing one or more metabolic products or intermediates may be identified.

Aspects of the invention take advantage of nucleic acid assembly technology that supports the production of any nucleic acid fragments (including large nucleic acid fragments) having a predetermined sequence of interest. Technology described herein allows nucleic acid and cellular libraries of the invention to be designed and assembled to include many different genetic elements of interest. This assembly technology also allows the production of nucleic acids that can be used to modify host organisms as described herein.

Thus, in one aspect, the invention provides a method of altering a cell function comprising introducing into a cell a nucleic acid comprising a set of genetic elements having recombination sites situated therebetween, rearranging the genetic elements by recombination at the recombination sites, and screening the cell for an altered cell function.

In some embodiments, the cell has been modified to delete genomic recombination sites. The genomic recombination sites may be reduced by 10-20%, 20-30%, 30-40%, 40-50%, 50-60%, 60-70%, 70-80%, 80-90% or 90-100%. In some embodiments, the genomic recombination sites are reduced by 50% or more. In some embodiments, the genomic recombination sites are reduced by 90% or more.

In some embodiments, the cell is a bacterial cell such as but not limited to an E. coli cell. In some embodiments, the cell is a eukaryotic cell such as but not limited a yeast cell, an insect cell, or a mammalian cell.

In some embodiments, the genetic elements are coding sequences. In some embodiments, the genetic elements are regulatory sequences. In some embodiments, the genetic elements are regulatory sequences and coding sequences. In some embodiments, the genetic elements are introns, in others they are exons, and in still others they are introns and exons.

In some embodiments, the method further comprises isolating the cell having an altered cell function.

In some embodiments, the nucleic acid is a vector. In some embodiments, the vector comprises a selection sequence. In some embodiments, the nucleic acid is integrated into the genome of the cell.

In some embodiments, the recombination sites are identical. In other embodiments, the recombination sites comprise at least two different types of recombination sites.

In some embodiments, the recombination sites are restriction enzyme sites. In some embodiments, the recombination sites are homologous recombination sites. In some embodiments, the recombination sites are susceptible to single or double stranded cuts.

In another aspect, the invention provides a method of producing a cell having an altered cell function comprising introducing into a cell a nucleic acid comprising a set of genetic elements having recombination sites situated therebetween, rearranging the genetic elements by allowing recombination between recombination sites, and isolating a cell having an altered cell function. In some embodiments, the method further comprises propagating the cell having an altered function.

In another aspect, the invention provides a method for producing a recombined nucleic acid molecule comprising producing a cell according to the method described above, and harvesting from the cell a recombined nucleic acid.

In some embodiments, the target nucleic acid or a product thereof (e.g., a recombined nucleic acid) may be amplified, sequenced or cloned after it is made. In some embodiments, a host cell may be transformed with the assembled target nucleic acid. The target nucleic acid may be integrated into the genome of the host cell. In some embodiments, the target nucleic acid may encode one or more polypeptides. The polypeptide may be expressed (e.g., under the control of an inducible promoter). The polypeptide may be isolated or purified (e.g., from a cell or cell lysate). A cell transformed with an assembled nucleic acid may be stored, shipped, and/or propagated (e.g., grown in culture).

In another aspect, the invention provides methods of obtaining target nucleic acids for generating biological diversity by sending sequence information and delivery information to a remote site. The sequence may be analyzed at the remote site. The starting nucleic acids may be designed and/or produced at the remote site. The starting nucleic acids may be assembled in a reaction involving a combination of ligation and extension techniques at the remote site. In some embodiments, the starting nucleic acids, an intermediate product in the assembly reaction, and/or the assembled target nucleic acid may be shipped to the delivery address that was provided.

Other aspects of the invention provide systems for designing starting nucleic acids and/or for assembling the starting nucleic acids to make a target nucleic acid. Other aspects of the invention relate to methods and devices for automating a multiplex oligonucleotide assembly reaction to produce libraries for generating biological diversity. Yet further aspects of the invention relate to business methods of marketing one or more methods, systems, and/or automated procedures that involve constructs for generating biological diversity.

Other features and advantages of the invention will be apparent from the following detailed description, and from the claims. The claims provided below are hereby incorporated into this section by reference.

### BRIEF DESCRIPTION OF THE FIGURES

FIG. 1 illustrates non-limiting aspects of an embodiment of a polymerase-based multiplex oligonucleotide assembly reaction;

FIG. 2 illustrates non-limiting aspects of an embodiment of sequential assembly of a plurality of oligonucleotides in a polymerase-based multiplex assembly reaction;

FIG. 3 illustrates a non-limiting embodiment of a ligase-based multiplex oligonucleotide assembly reaction;

FIG. 4 illustrates several non-limiting embodiments of ligase-based multiplex oligonucleotide assembly reactions on supports;

FIG. 5 illustrates a non-limiting embodiment of a nucleic acid assembly procedure; and,

FIG. 6 illustrates non-limiting embodiments of recombination products.

### DETAILED DESCRIPTION OF THE INVENTION

Aspects of the invention relate to methods and compositions for generating functional diversity and for identifying novel biological functions. In some aspects, the invention provides a set of genetic elements associated with recombination sites in an initial configuration (e.g., a vector comprising a linear array of genetic elements alternating with recombination sites). The recombination sites can promote rearrangement of the genetic elements thereby generating a plurality of different new configurations. Genetic elements may be genes, gene fragments, operons, subsets of genes from an operon, exons, introns, regulatory sequences, or other genetic elements that can confer a functional property (e.g., alone or in combination with one or more additional genetic elements). Accordingly, rearrangement of the genetic elements provides novel genetic configurations that may have new functional properties.

In some aspects, the invention provides methods for generating functional diversity in vivo by providing a population of cells containing an initial configuration of genetic elements associated with recombination sites and allowing or promoting recombination to generate a plurality of rearranged configurations of the genetic elements. Different rearranged configurations will be present in different cells. Appropriate selection and/or screening techniques may be used to identify cells that have a novel biological function of interest. The rearranged configuration of genetic elements that is associated with a novel biological function may be identified and/or isolated.

In some aspects, a cell line may be modified to remove one or more recombination sites (e.g., by deletion or alteration) from its genome. Such a modified cell line may be used as a chassis that can host different initial sets of genetic elements that are configured with the one or more recombination sites that were removed from the host genome. A lack of recombination sites on the host genome reduces the frequency of recombination between the set of genetic elements and the genome, thereby limiting recombination to rearrangements between the genetic elements of interest.

In one aspect, the invention may be used to generate and identify novel biological pathways, including, for example, novel regulatory pathways, metabolic pathways (e.g., catabolic or anabolic), or other novel biological pathways. In another aspect, proteins or RNAs with novel or modified functions may be generated and identified. In yet another aspect, methods of the invention may be used to modify existing biological pathways (e.g., to increase or decrease certain functions, to increase or decrease the accumulation of one or more intermediates or products, etc.).

Further aspects of the invention provide modified host cells that are designed to harbor libraries of the invention and allow for rearrangement of the genetic elements within the library without involving any rearrangement of the host genome. In some embodiments, a host genome may be genetically modified to remove one or more sequences in its genome that are identical or similar to the recombination sites in the library. For example, a host genome may be modified to remove one or more restriction sites that are used to promote recombination between different genetic elements within a library. Accordingly, a modified host cell of the invention can serve as a chassis for generating functional diversity from an appropriate library of initial nucleic acids that is introduced into the cell.

In aspects of the invention, recombination may result from the actions of endogenous host agents (e.g., nucleic acids, proteins, combinations thereof, and the like). In other embodiments, a host cell may be modified to express one or more agents that promote recombination between recombination sites. These agents are referred to herein as recombination inducing agents. Examples include recombination enzymes, restriction enzymes, topoisomerases, repair enzymes, and the like. In one illustrative embodiment, a host cell may be modified to express a restriction enzyme that acts on a recombination site. In another illustrative embodiment, a host cell may be modified to express one or a set of recombination enzymes that act on repeated sequences that are included in the initial nucleic acid library and/or that are introduced into the genome of the cell.

It should be appreciated that genes encoding recombination promoting agents should be expressed at suitable levels. Such levels promote a sufficient rate of genetic rearrangement (e.g., sufficient to provide a large pool of candidate configurations that can be screened or selected for new functions of interest). However, the rate of rearrangement should not be so high that the configurations are too unstable to be screened, selected, or maintained for subsequent analysis and/or propagation.

In some embodiments, genes encoding recombination promoting agents may be inducible thereby temporally limiting rearrangement to times when the genes are induced. In other embodiments, these genes may be constitutively expressed thereby promoting continuous rearrangement during cell growth.

Accordingly, aspects of the invention provide new methods for manipulating genetic elements (e.g., operons, genes, gene fragments, promoters, exons, introns, etc.) thereby opening up new opportunities to modify structure, function and temporal or spatial expression of proteins, protein function, metabolic pathways, and other cellular functions. Assembly methods of the invention can be used to generate any predetermined linked set of genetic elements and recombination sites in any initial configuration of interest. These initial configurations may be incorporated into vectors and/or introduced directly into host cells.

### Genetic Elements

As described herein, a genetic element may be any nucleic acid sequence that confers a biological property of interest (e.g., a biological property that may be altered through rearrangement with other genetic elements to obtain a new or modified biological property of interest). A genetic element may be a coding or a non-coding sequence.

In some embodiments, a genetic element is a nucleic acid that codes for an amino acid, a peptide or a protein. Genetic elements can be as short as a one or a few codons (e.g., a start codon). A genetic element may consist of an entire open reading frame of a protein, or it may consist of the entire open reading frame and one or more (or all) regulatory sequences associated with that open reading frame. Regulatory sequences include but are not limited to promoters, enhancers, silencers, transcriptional attenuation sequences, and the like. Genetic elements may be exons, introns, or nucleic acid sequences comprising both exons and introns. A genetic element can comprise a plurality of coding sequences and/or regulatory sequences. In some embodiments, a genetic element may be one or more regulatory and/or one or more coding sequences from a naturally-occurring operon (e.g., those found in bacterial sequences).

In some embodiments, nucleic acids that can adopt a particular secondary structure may be genetic elements. An example of such a nucleic acid is a poly-G sequence. As another example, a genetic element may be a nucleic acid having a sequence that induces polymerase slippage.

The genetic elements are linked together, preferably with recombination sites therebetween. As used herein, linked refers to a covalent bond between genetic elements and recombination sites. The covalent bond in its simplest form is a phosphodiester backbone of the nucleic acid molecule which comprises the genetic elements and recombination sites. Other linkages are also possible provided they do not interfere with the recombination of genetic elements and ultimately the transcription of the recombined nucleic acid.

The nucleic acids may further comprise mRNA stability and/or stabilization sequences. The location of these sequences may similarly be rearranged and thus they too may be genetic elements.

As described herein, genetic elements may encode known components of a pathway, candidate components of a pathway, operon components, groups of related enzymes, groups of enzymes that are candidates for rearrangement into a configuration that may have a biological activity or property of interest.

Constructs may be designed and assembled to contain a plurality of genetic elements that each expresses one or more different versions of any type of polypeptide (e.g., linear polypeptides, constrained polypeptides, and variants thereof) and/or variants containing a similar polypeptide scaffold, and or fragments thereof. According to the invention, such constructs can be used to generate new configurations that may have desirable novel or modified properties of the original polypeptide or scaffold that can be identified (e.g., using an appropriate screen or selection). A polypeptide scaffold may be based on, but is not limited to, one of the following peptides: cysteine-rich small proteins (e.g., toxins, extracellular domains of receptor proteins, A-domains, etc.), Zinc fingers, immunoglobulin-like domains (including, for example, the tenth human fibronectin type III domain and other fibronectin type III domains), lipocalins, lectin domains (including, for example, C-type lectin domain), ankyrins, human serum proteins (including, for example, human serum albumin), antibodies and antibody fragments (including, for example, single-chain antibodies, Fab fragments, single-domain (VH or VL) antibodies, camel antibody domains, humanized camel antibody domains), antibody regions (including one or more framework regions, one or more constant regions, one or more variable regions, one or more CDR regions), enzymes (including, for example, glucose isomerase, cellulase, hemicellulase, glucoamylase, alpha amylase, subtilisin, lipases, dehydrogenases, etc.), DNA-binding proteins (including, for example, the lac repressor, trp repressor, tet repressor, CAP activator, etc.), cytokines (including, for example, IL-1, IL-4, IL-8, etc.), hormones (including, for example, insulin, growth hormone, etc.), other suitable proteins, or combinations thereof.

In some embodiments, constructs contain genetic elements that express different scaffold polypeptides, or fragments thereof, having a specific biological function. Examples of biological functions are binding, inhibiting a biological process, catalyzing a specific reaction, etc. Non-limiting examples of scaffold polypeptides having a specific biological function are polypeptides that can bind to a linear polypeptide and polypeptides that can bind to a phosphotyrosine. Scaffolds of polypeptides that bind linear peptides can be based on proteins that are evolved to bind linear polypeptides. These proteins include major histocompatibility complex proteins (MHC I and MHC II), peptide transporter proteins, chaperones, proteases, and multi-domain proteins comprising peptide-binding domains such as poly(A)-binding protein, SH2 domains, SH3 domains, PDZ domains, and WW domains. Major histocompatibility complex proteins display peptides of 9-12 amino acids on the surface of antigen-presenting cells, where the MHC-peptide complex can be recognized and bound by T-cell receptor. Humans have several hundred different MHC alleles, which vary in their specificity and affinity for specific peptides. MHC polypeptide scaffolds are designed based on the analysis of theses alleles. Peptide transporter proteins bind to linear peptides of 2-18 amino-acid residues, and bury at least a part of the peptide in their core. The transporter-peptide complex can subsequently be translocated across the membrane with the help of additional transport complex components. One example of a peptide transporter is the oligopeptide permease (Opp) family, with different members of the family recognizing peptides of different lengths and sequences with nanomolar to micromolar affinity. One member of the family, the Opp protein of Lactococcus lactis (OppAL1) can bind and transport peptides of up to 18 residues and longer. Polypeptide scaffolds are designed based on the analysis of the peptide binding properties, including the core region, of OppAl1 and other peptide transport proteins. Proteases cleave polypeptides, and differ widely by their degree of substrate specificity. Inactive mutants have been constructed that bind polypeptides, but do not cleave them. These mutant proteases are therefore particularly suited as scaffold polypeptides for polypeptides with peptide binding properties. The poly(A)-binding protein (PABC) has a C-terminal domain of interacts with translational factors in a random-coil configuration. The peptide motif that binds to PABC comprises 12-15 amino-acid residues and is in a formation resembling random-coil when bound to PABC. The peptide binding domain of PABC of various species can be analyzed to identify residues essential to peptide binding. Scaffold polypeptide for libraries of peptide binding polypeptides are designed based on these principles.

Scaffolds of polypeptides that bind phosphotyrosines can be based on proteins that are evolved to bind and/or process phosphotyrosines. Phosphotyrosine binding and processing proteins include proteins with phosphotyrosine-binding (PTB) domains, protein tyrosine phosphatases (PTPs), and mitogen-activated protein kinase (MAPK) phosphatases (MKPs). Phosphotyrosine-binding (PTB) domains are naturally occurring phosphotyrosine binding modules. The protein structure generally falls under the pleckstrin homology (PH) superfold. The peptides are recognized in general according to the motif N-P-X-(phosphoY/Y/F) whit the peptide binding as a type I beta turn. Examples of mammalian PTBs include Shc, Sck, X11, Doc-2, and p96, while drosophila PTBs include Dab and Numb. There are at least 50 PTB domains known from at least 46 proteins, with many structures elucidated by NMR or crystal structures, for instance Shc, X11, IRS-1, Talin, Dab1/2, Numb, SNT, Dok1/5, Radixin, and tensinl. Proteins with PTBs are analyzed to design a scaffold polypeptide for phosphotyrsosine binding. Extra weight will be given to proteins that bind phosphotyrosine peptides in a phosphotyrosine dependent manner. Examples of such proteins include Shc-like PTBs, and IRS-like PTBs (which include IRS, Dok, and SNT) and proteins including the C2 domain of PKCδ and possibly PKCθ. Protein tyrosine phosphatases (PTPs) often play a critical role in cellular regulation by dephosphorylating tyrosines of signaling molecules. PTPs include both receptor-like PTPs and non-transmembrane PTPs. Some examples of PTPs are SHP-2 (PTPN11), PTP-1B (PTPN1), TCPTP (PTPN2), PEP (PTPN22), SHP-1 (PTPN6), PTP-PEST (PTPN12), PTP-MEG2 (PTPN9), STEP (PTPN5), and HePTP (PTPN7). While PTPs process phopshotyrosine peptides, the phosphatase activity can be inactivated resulting in a polypeptide that can bind phosphotyrosines but can not process them. The PTP active site generally contains the motif HC(X₅)R, and may additionally contain a WPD motif. The dephosphorylation function can be inactivated by introducing one or more mutations in the active-site. For example, the essential C and/or R (such as C-S), and/or the invariant D (such as D-A), or combinations thereof (such as C-S/D-A or D-A/Q-A) can be mutated to result in an inactive phosphatase activity. Scaffold polypeptides are designed based on these inactivated PTPs. Mitogen-activated protein kinase (MAPK) phosphatases (MKPs) are related to PTPs and can dephosphorylate both phosphothreonine and phosphotyrosine residues. MPKs are found in various mammalian pathways, including ERK, JNK (MAPK8), p38 (MAPK14). The active site of these proteins is mutated to result in a polypeptide scaffold and this polypeptide scaffold san subsequently be used as a scaffold for a library of phosphotyrosine binding polypeptides.

### Recombination Sites

As used herein, a recombination site is a nucleotide sequence that induces or facilitates recombination in vitro and/or in vivo. In many instances the site is recognized, bound by, and/or acted upon by a recombination promoting agent such as a protein.

In some embodiments, a recombination site is a restriction enzyme site (i.e., a site recognized by and/or cleaved by a restriction enzyme). After cleavage by a restriction enzyme, a restriction site can promote recombination. Restriction sites may be of any length (e.g., 4-20 base pairs). The longer the restriction site, the less frequently it will normally occur in a genome. Enzymes that cut these longer sequences are sometimes referred to as “rare cutters”. Suitable restriction enzyme sites may be found, for example, in a commercial catalog (e.g., New England Biolabs). Most restriction enzymes will induce a double strand break. However, the action of certain restriction enzymes will result in a single strand nick only. A single strand nick also may promote recombination because the processing of this nick by a replication fork or DNA repair enzymes can induce a recombination event. It should be appreciated that for a restriction site to act as a recombination site in vivo, the appropriate restriction enzyme must also be present in the cell. The enzyme may be endogenous to the cell or may be ectopically expressed or introduced into the cell directly as a protein.

In certain embodiments, a recombination site is a sequence-specific recombination site (e.g., a lox P site) that is recognized by a recombinase (e.g., the Cre enzyme). It also should be appreciated that for a sequence-specific recombination site to act as a recombination site in vivo, the appropriate recombinase enzyme must also be present in the cell. The enzyme may be endogenous to the cell or may be ectopically expressed or introduced into the cell directly as a protein.

In some embodiments, any repeated nucleic acid sequence can be a recombination site. For example, any nucleotide sequence can be a recombination site if there are two or more identical or homologous nucleotide sequences interspersed between genetic elements. Since recombination is promoted by homology, a greater homology (e.g., either in length or percentage) promotes a higher recombination frequency. Preferably, these types of recombination sites share 100% identity (i.e., their nucleotide sequences are identical). However, homologous recombination can also occur between sequences that have not identical yet still share a high degree of homology. Thus these sequences may share greater than 50%, 60%, 70%, 80%, 85%, 90%, 95%, 96%, 97%, 98% or 99% homology. The entire nucleotide sequence located between consecutive genetic elements may be a recombination sequence, or only a fragment thereof may be. The nucleotide sequences located between genetic elements may determine their propensity to participate in desired recombination events. For example, a particular recombination site can be designed to recombine specifically with only one other recombination site. This can be accomplished if the two sites have sequences that are rare and highly homologous if not identical. In some embodiments, recombination sites can be designed to recombine with many other locations by using sequences that are identical or highly homologous to sequences that occur frequently.

Examples of recombination enzymes include but are not limited to tyrosine recombinases, serine recombinases, Flp, RecA, Pre (plasmid recombination enzyme) and ERCC1.

In some embodiments, recombination can be induced by certain nucleotide modifications or processes. For example, DNA strand breaks (e.g., double strand breaks and/or single strand breaks) can promote recombination. Damaged or modified bases, or abasic sites also can induce recombination. Any nucleotide modification that results in the stalling of a replication fork also can induce recombination. Accordingly, modified or damaged nucleotides can be recombination sites, as can sites acted upon by enzymes that modify and/or damage nucleic acids in this manner.

In certain embodiments, a recombination site is any stretch of nucleotides that can induce recombination through a triggering event. For example, bases that are susceptible to modification may be recombination sites. Such bases, when modified, can be removed by repair enzymes or through a physical action (e.g., exposure to heat or light). Removal of damaged bases produces abasic sites that can induce recombination. In some embodiments, if multiple damaged sites are located opposite from each other, removal of damaged bases can lead to DNA double strand breaks that also promote recombination.

The linked set of genetic elements having recombination sites situated therebetween may utilize a single type of recombination site, such that recombination between any and all genetic elements may occur with an approximately equal probability. In other instances, the linked set of genetic elements may utilize two or more types of recombination sites. In these latter instances, there should be at least two copies of each recombination site so that each site has at least one recombination partner. In these embodiments, the initial nucleic acid is designed to increase the recombination frequency between particular genetic elements while almost precluding these recombination with other genetic elements.

In some embodiments, recombination sites can be recognition sites or motifs for one or more nucleic acid modification enzymes (e.g., methylases and/or other enzymes) that can specifically modify a sequence in a way that promotes recombination (e.g., by damaging one or more bases, e.g., by alkylating one or more bases, generating one or more abasic positions, other modifications, or any combination thereof).

### Vectors

Initial configurations of genetic elements and recombination sites may be provided in the form of a single or double-stranded linear or circular nucleic acid molecule with or without vector sequence. These initial configurations are referred to herein as initial nucleic acids. In some embodiments, an initial configuration of genetic elements and recombination sites may be cloned into a vector. A vector may be any suitable vector. For example, a vector may be a plasmid, a cosmid, a phagemid, a BAC, a YAC, an F factor, or any other suitable prokaryotic, eukaryotic or viral vector. A vector may include an origin of replication and/or one or more selectable markers (e.g., antibiotic resistant markers, etc.) and/or detectable markers (e.g., fluorescent markers, etc.). In some embodiments, a vector may be a shuttle vector that is functional in two or more different types (e.g., species) of host cells.

It should be appreciated that a vector may be selected or modified to remove recombination sites that could interfere with the desired recombination events involving the recombination sites that are being used to promote rearrangements of the genetic elements of interest. Vectors may therefore be modified to reduce the number of one or more recombination sites by at least 10%, 20%, 30%, 40%, 50%, 60%, 70%, 80%, 90%, 95% or by 100%.

Vectors can be introduced into a host cell through a variety of mechanisms. They can be transformed, transfected or introduced by physical techniques like microinjection or electroporation. In other embodiments, vectors may be introduced through biological means, for example using phages or viruses. Many methods of introducing oligonucleotides into cells are known to persons of ordinary skill in the art and are incorporated herein by reference.

Upon introduction of an initial nucleic acid (whether or not in the context of a vector) into a host cell, the genetic elements can undergo recombination. Recombination can be initiated by replication-associated events, or through other triggering events such as the initiation of DNA strand breaks in the recombination sites through the action of restriction enzymes or the creation of DNA strand breaks through other means.

In some embodiments, a low copy number vector (e.g., plasmid) may be used to maintain the initial linked set of genetic elements and recombination sites and avoid a potential loss of elements due to toxicity or other issues that may be associated with high copy number vectors.

In some embodiments, an initial set of genetic elements and associated recombination sites may be integrated into the genome of the host cell. This may involve integrating a vector into the genome of a host cell. Accordingly, a host cell and/or a plasmid may be modified to introduce a homologous sequence that could promote integration of the plasmid into the genome. The plasmid may be replication defective in the host that is being used to generate the rearranged configurations of genetic elements (i.e., the target nucleic acids). By incorporating the genetic elements and recombination sites into the host genome, multiple recombination events can occur without losing the vector or needing to select for the vector. Also, in some embodiments the set of genetic elements and recombination sites may be more stable if they are integrated as a single copy into the genome of the host.

### Host Cells

Any cell type may be suitable as a host cell provided it can perform the recombination functions required for rearrangement of the genetic elements. The cell may be inherently or endogenously capable of such recombination or it may be manipulated to be so. In some embodiments, a host cell expresses one or more restriction enzymes and/or one or more recombinase enzymes that can act on one or more of the recombination sites being used to generate rearrangements. The enzyme may be encoded in a vector or in the genome of the host cell (e.g., the gene encoding the enzyme may be integrated into the genome of the host cell). In some embodiments, expression of the enzyme can be controlled (e.g., inducible). For example, the gene encoding the enzyme can be placed under the control of a specific promoter. This can be used to control the timing and duration of recombination by turning enzyme expression on or off when appropriate. Accordingly, the extent of recombination to be controlled. By switching off enzyme expression, a pool of rearranged configurations (i.e., target nucleic acids) can be maintained in a stable form and exposed to appropriate selection and or screens for a biological function of interest.

In some embodiments, a host cell may be modified to provide a platform or chassis that can be used for multiple screens or selections starting with different sets and/or configurations of genetic elements. The genome of a host cell may be modified to remove sequences that can induce unwanted recombination.

In some embodiments, to accommodate multiple rounds of recombination events between sites on one or more vectors that are introduced into a host cell, the genome of the cell may be modified to remove recombination sites that potentially may interfere with intra- or inter-vector recombination. In certain embodiments, the chassis-cell will be engineered to have no recombination sites at all. In other embodiments, the chassis will have a subset of its naturally occurring recombination sites removed (e.g., about 10%, 20%, 30%, 40%, 50%, 60%, 70%, 80%, 90%, or more).

Any restriction site may be used as a recombination site and may therefore be removed (completely or some fraction thereof) from the genome of a host cell. In some embodiments, rarer restriction sites may be selected (e.g., ones that recognize a unique long site). In some embodiments, one or more 4 base cutters or 6 base cutters recognition sequences may be removed. In some embodiments, the recognition sequences of one or more of I-SceI, I-CeuI, PI-PspI, PI-SceI, and NotI restriction sites may be removed. In some embodiments, CTAG sites may be removed from the genome of the host and used as part of a recombination site in association with the set of genetic elements.

It should be appreciated that certain or all recombination sites can be removed from the genome without a penalty if the site is essential to the genome (e.g., if it is a non-transcribed sequence). If one or more recombination sites is in for example an actively transcribed part of the genome, and cannot be removed without compromising the viability of the cell, its ability to act as a recombination site may be reduced or eliminated by mutation. For example, if the site is a homologous recombination site then the site may be mutated by reducing the level of identity or homology to the point where it would no longer recombine with the recombination sites in between the genetic elements of the initial nucleic acid. If the site is in a coding region, it may be mutated by using alternate codons, and thereby not affecting the protein sequence. If the recombination sites of the vector(s) are based on restriction sites that need to be activated, genomic restriction sites having the same sequence can be removed or inactivated to avoid or reduce the frequency of vector-genome recombination.

In some embodiments, a “chassis” cell may be modified to remove all (or a subset, including for example at least 10%, 20%, 30%, 40%, 50%, 60%, 70%, 80%, 90% or more) of two or more (e.g., 2, 3, 4, 5, 6, 7, 8, 9, 10 or more) different recombination sites. For example, two or more different restriction sites may be removed. These cells also may be modified to express two or more different restriction enzymes that recognize these sites. These enzymes may be independently inducible. These cells may be used to promote recombination of different sets of genetic elements that are associated with different restriction sites. In some embodiments, an initial configuration of a set of genetic elements may include two or more different restriction sites (e.g., distributed in the same configuration or in different configurations). A chassis that expresses the corresponding restriction enzymes under different regulatory controls can be used to promote independent rearrangement of different components of the initial set of genetic elements by expressing different restriction enzymes.

In some embodiments, the genome of the host may be modified to introduce a sequence that is homologous to a sequence on a vector or other nucleic acid containing the set of genetic elements and recombination sites in order to help integrate them into the genome of the host cell. In some embodiments, the genome of a host cell may be modified to provide recombination sequences to allow genomic integration of two or more different sets of genetic elements.

In some embodiments, the genome of a host cell may be reduced (e.g., by 5%, 10%, 15%, or more) in order to accommodate the sets of genetic elements being integrated.

A host cell may be prokaryotic (e.g., bacterial such as E. coli or B. subtilis) or eukaryotic (e.g., a yeast, mammal or insect cell). For example, host cells may be bacterial cells (e.g., Escherichia coli, Bacillus subtilis, Mycobacterium spp., M. tuberculosis, or other suitable bacterial cells), yeast cells (for example, Saccharomyces spp., Picchia spp., Candida spp., or other suitable yeast species, e.g., S. cerevisiae, C. albicans, S. pombe, etc.), Xenopus cells, mouse cells, monkey cells, human cells, insect cells (e.g., SF9 cells and Drosophila cells), worm cells (e.g., Caenorhabditis spp.), plant cells, or other suitable cells, including for example, transgenic or other recombinant cell lines. In addition, a number of heterologous cell lines may be used, such as Chinese Hamster Ovary cells (CHO). It should be appreciated that when integrating a nucleic acid into a eukaryotic genome (e.g., a mammalian genome) care should be taken to select sites that will allow sufficient expression (e.g., silenced regions of the genome should be avoided, whereas a site comprising an enhancer may be appropriate).

In some embodiments, a host cell may be selected for its recombination properties. In some embodiments, a host cell may be selected for its metabolic properties. For example, if a selection or screen is related to a particular metabolic pathway, it may be helpful to use a host cell that has a related pathway. Such a host cell may have certain physiological adaptations that allow it to process or import or export one or more intermediates or products of the pathway. However, in other embodiments, a host cell that expresses no enzymes associated with a particular pathway of interest may be selected in order to be able to identify all of the components required for that pathway using appropriate sets of genetic elements and not relying of the host cell to provide one or more missing steps.

Examples of organisms that may have useful phenotypes include, but are not limited to Deinococcus radiodurans and Vibrio furnissii. Deinococcus radiodurans has evolved a strong capability for recombination and introduced vectors can be recombined at high frequency. V. furnissii can produce n-alkenes from products found in waste-water and is commercially interesting. (Park et al., 2005, J. Appl. MicroB. 98, 324). The bacterium already has a pathway in place for n-alkene synthesis.

The genome of a host organism may be modified through the re-synthesis of large parts of the genome and replacing the original genome (or a portion thereof) with a new optimized genome (or a portion thereof) through recombination. In some embodiments, assembly methods described herein may be used to generate these large genome parts.

In some aspects of the invention, cells may be modified to add recombination elements between naturally occurring genomic genetic elements (e.g., between predetermined genomic elements of interest). Recombination within such cells also generates functional diversity that can be used to screen or select for one or more novel functions of interest. This approach may be particularly useful if the host cell genome encodes an operon or other cluster of genes selected for analysis.

It should be appreciated that a host cell may be a cell that naturally expresses, or is engineered to express, sufficient levels of one or more recombination genes (e.g., RecA, etc.), and/or modification genes (e.g., gene(s) encoding one or more nucleic acid methylases or other enzymes that can modify nucleic acids in a way that may promote recombination), and/or error correction genes that promote recombination. In some embodiments, one or more of these genes may be inducible so that levels of recombination can be regulated.

### Configuration of Sets of Genetic Elements and Recombination Sites

Aspects of the invention may involve any combination of any appropriate number of genetic elements and recombination sites. For example, 2-5, 5-10, 10-20, 20-50, 50-100 or more different genetic elements may be included. Each genetic element may be flanked by two recombination sites resulting in a configuration of alternating genetic elements and recombination sites. However, other configurations may be used. For example, several genetic elements may be grouped together and not separated by recombination sites (e.g., if they perform a core function to the desired biological function being screened or selected for). In some embodiments, the genetic elements are genes (including sequences required for transcription and translation). In some embodiments, the genetic elements are part of a natural operon and are under transcriptional control of a single promoter. In some embodiments a plurality of different genetic elements may be separated by restriction sites but artificially brought under the control of a single promoter in an artificial operon. In some embodiments, the identity of the genetic elements that are included in the initial set may be determined by the type of biological function that is being selected or screened for. For example, if an improved or altered enzyme function is desired, multiple copies of a gene encoding the enzyme may be used, each copy having one or more sequence variations. The recombination sites may be designed to allow rearrangement of different regions of the gene so that different sequence combinations can be sampled. In contrast, if a new or modified metabolic pathway is desired, a plurality of different enzymes that have functions related to the desired pathway may be used along with different promoter and other regulatory sequences. Recombination sites may be placed between these different genetic elements so that different combinations of genes expressed at different levels may be sampled. It should be appreciated that combinations of these strategies may be implemented. It also should be appreciated that combinations of genetic elements from different organisms also may be grouped together in an initial set.

As discussed above, the recombination sites that flank the genetic elements can be the same or different. In another embodiment, multiple copies of the recombination site are inserted in the vector thereby increasing the likelihood of a recombination event. In other embodiments, genetic elements are flanked by different recombination sites. Having different recombination sites has the advantage that more than one recombination event can be triggered independently. Any combination of recombination sites (e.g., restriction sites, homologous sequences, etc.) can be used when assembling these different recombination sites.

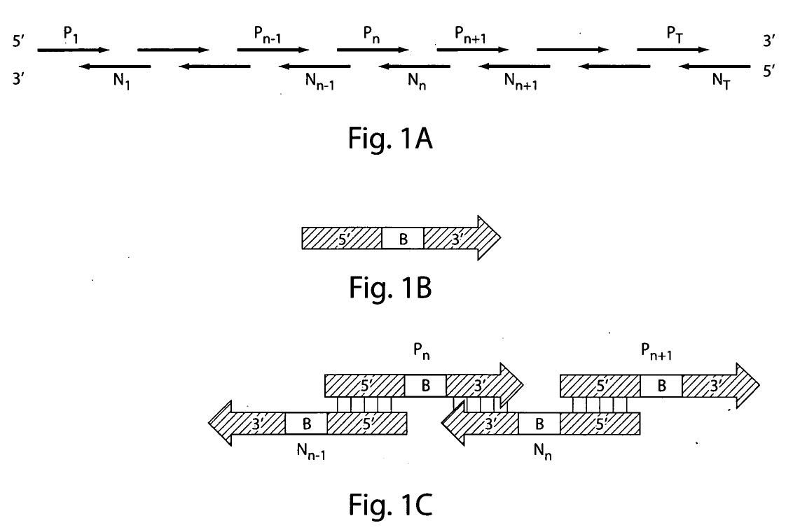

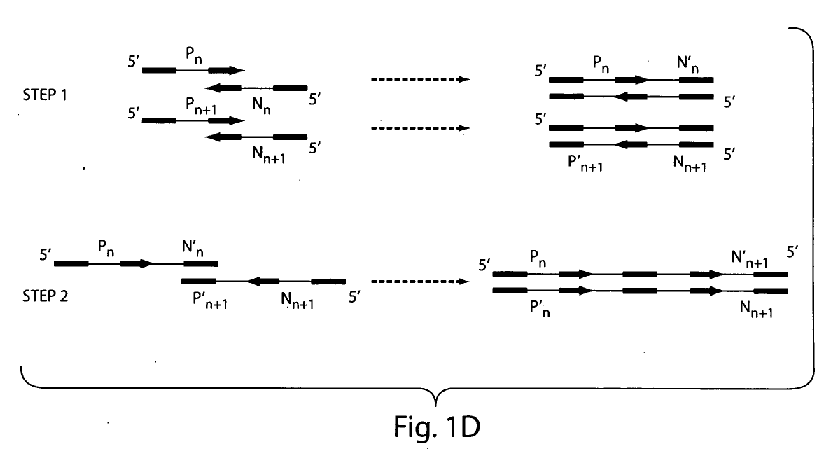

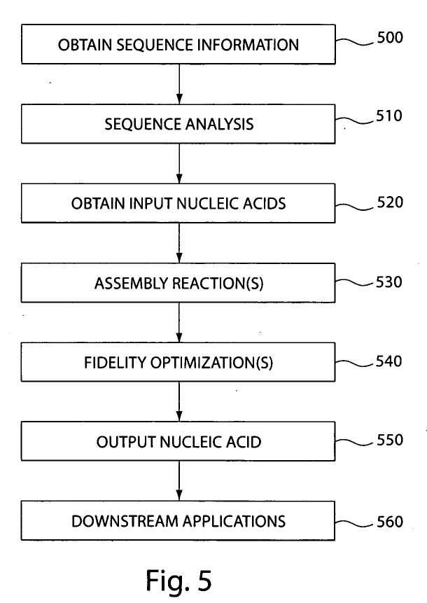

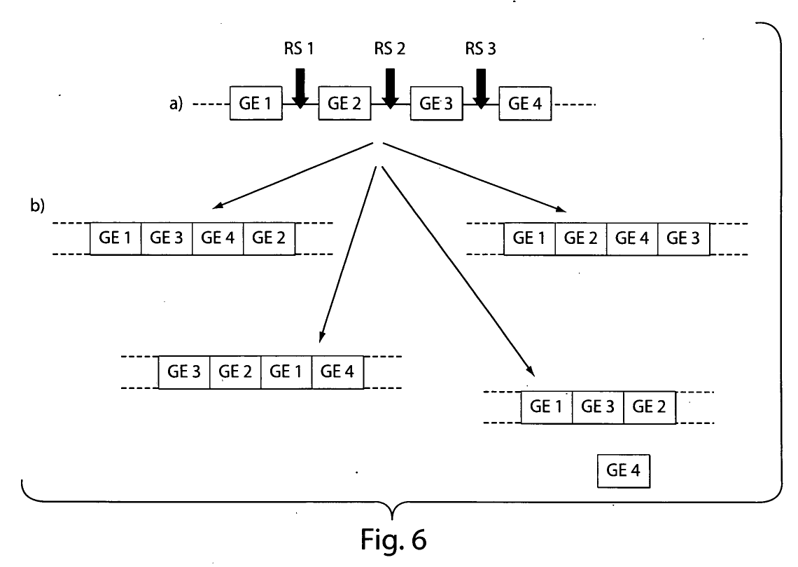

Aspects of the invention may be used in conjunction with in vitro and/or in vivo nucleic acid assembly procedures to generate starting constructs having a desired combination of genetic elements and recombination sites. Assembly techniques that can be used to prepare constructs of interest those illustrated in FIGS. 1-4. FIG. 5 illustrates a method for assembling a nucleic acid in accordance with one embodiment of the invention. FIG. 6 illustrates a non-limiting example of nucleic acid construct and the resulting recombination products that represent biological diversity. FIG. 6 a) provides an example of a construct containing genetic elements (GE 1-4) separated by recombination sites (RS 1-3). It should be appreciated that a construct may include a plurality of genetic elements and/or recombination sites. The genetic elements may all be different. However, in some embodiments, two or more copies of one or more genetic elements may be included. In some embodiments, the recombination sites are all similar or the same. However, two or more different recombination sites may be included. In some embodiments, all of the recombination sites may be different. A construct may include about 1, 2, 3, 4, 5, 6, 7, 8, 9, 10, 15, 20, 25, 30, 35, 40, 45, 50, 60, 70, 80, 90, 100 or more genetic elements and/or recombination sites. In certain embodiments, a construct may not include a recombination site between each genetic element, for example, in some constructs a recombination site may occur between every other genetic element. It should also be appreciated that a recombination event may occur resulting in recombination of a section of a construct where such a section of the construct includes more than one genetic element. In such an example, a recombination site may be located on either side of a region containing more than one genetic element such that the section as a whole is involved in the recombination event. FIG. 6b) provides an example of some of the possible recombination events that may occur from the illustrated construct. During cell division and/or growth the genetic elements may undergo recombination resulting in progeny cells having recombined nucleic acids. Cell division may result in several progeny cells each having different recombined nucleic acids. It should be appreciated that one or more recombination sites may remain in a progeny cell, for example one or more recombination sites may be carried forward during cell division. In such examples, a progeny cell may be capable of undergoing further recombination to form further recombined nucleic acids.

### Screens and Selections for Biological Functions

Under appropriate conditions, rearrangement of genetic elements is promoted in vivo inside a host cell due to the presence of the recombination sites. Rearrangement of the genetic elements provides novel genetic configurations that may have new functional properties. In some embodiments, the order of two or more of the genetic elements may be altered (e.g., inverted). In some embodiments, the relative position of a plurality of the genetic elements may be changed. In certain embodiments, one or more genetic elements may be deleted. In some embodiments, one or more genetic elements may be duplicated. One or more of the recombination sites may be lost during recombination. In some embodiments, recombination sites may be designed to be deleted either during recombination or during cell growth so that the rate of recombination reduces over time as the number of recombination sites are progressively lost. The resulting recombined products can thereby be stabilized. Methods for promoting progressive loss of recombination sites can include providing recombination sites that are or are surrounded by repeat sequences (e.g., direct or indirect) that are susceptible to loss. However, any suitable sequence (e.g., a toxic sequence) that is susceptible to progressive loss during cell growth may be associated with a recombination site in embodiments where progressive loss of the recombination sites is desired. The rate of loss of the recombination sites also may be determined by the recombination properties (e.g., level of activity) of the host cell. In some aspects, the invention provides methods for selecting or screening for novel functions. Host cells harboring the libraries of target nucleic acids (i.e., recombined nucleic acids) may be exposed to appropriate conditions to identify one or more novel functions of interest. Novel functions may include altered activities of existing enzymes, novel regulatory responses (e.g., altered patterns of response to a signal, response to a novel signal, etc., or combinations thereof), novel combinations of enzymes that result in novel pathways (e.g., novel metabolic pathways), other novel functions, or combinations thereof.

In some embodiments, selection or screening may be performed on the host cell in which genetic rearrangement occurred. In other embodiments sets of genetic elements are allowed to undergo recombination in chassis cells and are subsequently extracted from the chassis cells. The rearranged vectors can then be screened in vitro or can be introduced in an alternative cell line, which does not have to be a chassis cell, to be analyzed in vivo.

Aspects of the invention may be used for pathway engineering in vivo. The evolution of entire metabolic pathways rather than just one enzyme may be particularly useful because compounds are often produced or metabolized in a process involving multiple steps of a pathway rather than by one enzyme. Multi-enzyme pathways can also be engineered through the manipulation of certain key enzymes in the pathway.

Once a pool of rearranged genetic elements has been generated, the candidates can be used to screen or select for biological properties of interest. Candidates can be screened while recombination is still proceeding. In some embodiments, candidates can be screened after a certain number of recombination events have taken place.

Candidates can be screened for by selective pressure (e.g., whether the organism survives when a toxin is added to the growth environment or when an essential nutrient is removed, etc.). Further non-limiting examples of screening or selection techniques may include growing organisms at high temperatures or in organic solvents. If a specific enzyme is targeted for optimization, an enzyme-specific selection process can be used.

In some embodiments, metabolic pathways can be screened for in functional screens. Screening for metabolic pathway can involve screening for the occurrence of a desired final product or one or more intermediate products by using a reporter assay for the one or more products. Other non-limiting techniques that can be used may include monitoring decreases in precursor amounts, monitoring metabolism on a related fluorescent compound, and others.

In one embodiment, binding assays can be used to detect the synthesis of a desired end product. In one embodiment, the desired product is detected using a binding partner such as an aptamer. Aptamers can consist of DNA and/or RNA sequences. Aptamers that bind to metabolites, intermediates or a variety of other compounds can be used. An aptamer that binds a metabolite or intermediate of interest can be developed and used. Binding of the aptamer to the metabolite or intermediate can be assayed for with a reporter that can be an integral part of the aptamer. The reporter can also be a molecule that detects the difference between bound and unbound aptamer. It should be appreciated that a plurality of different aptamers (e.g., 2, 3, 4, 5, 6, 7, 8, 9, 10 or more) with different readouts may be used to monitor the levels of different metabolites or intermediates.

### Applications

Aspects of the invention may be used to synthesize higher levels of one or more predetermined metabolites, synthesize one or more new metabolites, synthesize altered combinations of metabolites, provide internal regulatory connections (e.g., in the form of feedback loops), provide external regulatory connections (e.g., for response to environmental factors, human factors, etc.), provide intracellular regulatory connection (e.g., between two or more metabolic pathways) provide signals that can be used to monitor one or more intermediate processes or metabolites, etc., or any combination thereof.

Aspects of the invention may be used for pharmaceutical applications (e.g., to provide engineered pathways that may be useful to gene therapy).

Aspects of the invention may be used for industrial applications (e.g., to provide engineered pathways that may be useful to increase the synthesis of a product of interest or to provide additional internal or external regulatory connections to regulate the synthesis of a product in response to different factors). Industrial products of interest may include industrial enzymes, metabolites that are useful as feedstocks for industrial syntheses, and other organic or biological products. Industrial products such as propanediol, octane, diesel fuel, ethanol, butanol, lactic acid, polymers, amino acids, polyhydroxybutyrate, alkaloids, terpenes, polyketides may also be of interest.

Aspects of the invention may be used for agricultural applications (e.g., to provide engineered pathways that may be useful to engineer crops to express one or more products of interest and/or to provide additional internal or external regulatory connections to regulate the synthesis of a product in response to different factors). In some embodiments, pathways may be engineered to increase photosynthetic yields of agricultural products (e.g., in vivo in plants). Pathways may also be engineered to increase aesthetic, odor, or other consumer appeal, or to ingest and/or digest environmental toxins. Products may include fruits, vegetables, grains, flowers, trees, shrubbery, canes, and reeds.

In some embodiments, pathways may be adapted to increased levels or scales of production of one or more metabolites (e.g., for agricultural, industrial, pharmaceutical, or other purposes). For example, additional regulatory components may be added (e.g., feedback or feedforward loops, regulatory components that are responsive to external stimuli, for example to induce a pathway at a desired time during production or at an appropriate time during an agricultural season, etc.).

Aspects of the invention also may be used to develop engineered pathways for environmental applications (e.g., for remediation by providing mixtures of functional components or engineered organisms that can metabolize one or more environmental contaminants to either sequestrate the contaminants and/or process the contaminants to form one or more environmentally acceptable compounds (e.g., less toxic). In some embodiments, pathways of the invention may be used for scavenging environmental contaminants and/or toxic compounds (e.g., as part of an environmental cleanup or remediation effort). In some embodiments, engineered pathways may be used to waste water treatment. In some embodiments, pathways and/or organisms may be engineered to increase absorption or incorporation of environmental toxins or pollutants (e.g., compounds dissolved in water, ground contaminants, air contaminants, carbon dioxide, carbon monoxide, sulfur, etc.).

Aspects of the invention also may be used for energy generation. In some embodiments, pathways may be developed to increase the production of a fuel or of a substrate for a industrial fuel processing technique. For example, unicellular or multicellular plants (e.g., algae, crop plants, grasses, trees, etc.) may be developed with engineered pathways to increase the yield of certain compounds or compound substrates. For example, pathways may be engineered to increase the yields of alcohols (e.g., methanol, ethanol, etc.), sugars, animal fats, vegetable oils, hydrocarbons such as isooctane or cetane, other combustible compounds, etc., or any combination thereof. In some embodiments, pathways may be engineered to increase photosynthetic yields of fuel substrates or products (e.g., in vivo in plants).

Aspects of the invention also may be used to provide one or more markers of pathway activity. A marker may be responsive to the level or status of a metabolite, a functional component, and/or a regulatory component. A marker may be for example, a binding moiety (e.g., a protein or a nucleic acid, for example, an aptamer) that is responsive (e.g., generates a color) to one or more indicators of pathway activity. The color for example may be generated by expression or activation of an engineered GFP or other protein reporter system.

Aspects of the invention also relate to providing cells that are engineered to include one or more different pathways. For example, a cell may be engineered to include several (e.g., 2, 3, 4, 5, or more) independent pathways or interdependent pathways that are connected via a regulatory network. In some embodiments, the level of one or more metabolites produced in a first pathway may provide positive or negative signal to one or more functional elements in a second pathway. In some embodiments, two or more pathways may be regulated by the same extrinsic signal(s). Different pathways may be alternative pathways for generating the same product(s). Different pathways may be alternative pathways for metabolizing the same substrate(s). However, different pathways may provide unrelated synthetic and/or catabolic functions. Accordingly, a multipurpose cell may be engineered that is responsive to a plurality of different signals and/or metabolites. In processes in which cells ferment mixtures such as natural sugars, it may be desirable to utilize a multipurpose cell that can convert all distinct sugar molecules present to a target end product. In some embodiments, the individual sugar molecules may be converted to product or utilized with different efficiencies, and it may be optimal to adjust the rate of consumption of substrates individually. In another embodiment, a multipurpose cell may be utilized to detect more than one molecule in its environment. The cell may respond in the same manner for each input molecule thus allowing it to be determine that at least one of a set of molecules is present, or it may respond in a different manner for each, thus allowing the specific molecules present to be identified individually. These may be responsive to different toxins or pollutants and either may process them to reduce their toxicity and/or provide a signal indicating their presence.

The invention therefore provides methods and compositions for generating cells having modified and in some instances novel function. These functions are essentially unlimited. Such functions arise from the synthesis of a new nucleic acid that imparts a particular biological function as a result of the order or its genetic elements. For example, a particular biochemical pathway in a cell may be altered as a result of a difference in the ratios of enzymes and substrates involved in the pathway. As another example, a particular signaling pathway in a cell may be altered as a result of a difference in the ratios of kinases, phosphatases, adaptors, and downstream transcription factors. The target nucleic acid (i.e., the final recombined product) can be isolated from the chassis cell and introduced into another cell that is for example amenable to the particular desired function. The target nucleic acid may be integrated into the host cell genome or it may exist as an extragenomic plasmid or vector.

Cells comprising these new pathways therefore find wide application including environmental applications such as petroleum metabolism, degradation and/or conversion, pollutant metabolism, degradation and/or conversion, toxic waste metabolism, degradation and/or conversion, greenhouse gas metabolism, degradation and/or conversion, ethanol production, ethanol conversion, synthesis of novel compounds including biologics, altered enzymes, and the like; agricultural applications such as manure metabolism, degradation and/or conversion, methane metabolism, degradation, conversion and/or capture, corn degradation and conversion (e.g., into ethanol), generation of microbe resistant plants or crops, generation of faster growing or faster maturing plants or crops, generation of plants or crops with particular phenotypes including altered color, smell, taste and the like; food industry application such as generation of faster fermenting yeast for the bread industry, generation of more stable bacteria for the cheese and milk industry; biotechnology applications including increased synthesis of biochemical products such as nucleotides, amino acids, proteins, enzymes, and the like; generation of altered protein complexes such as proteosomes, inflammasomes, transcriptional machinery and complexes, and the like.

Aspects of the invention may involve one or more nucleic acid assembly reactions in order to make the sets of genetic elements and recombination sites, the modified host cells, the aptamers, and/or other nucleic acids that may be used to generate biological diversity and screen or select for one or more functions of interest.

Aspects of the invention may be used in conjunction with in vitro and/or in vivo nucleic acid assembly procedures. Assembly strategies of the invention include those illustrated in FIGS. 1-4. FIG. 5 illustrates an example of nucleic acid assembly procedure that can be used to design and assemble a construct of the invention. Initially, in act 500, sequence information is obtained. The sequence information may be the sequence of a predetermined target nucleic acid that is to be assembled. In some embodiments, the sequence may be received in the form of an order from a customer. The order may be received electronically or on a paper copy. In some embodiments, the sequence may be received as a nucleic acid sequence (e.g., DNA or RNA). In some embodiments, the sequence may be received as a protein sequence. The sequence may be converted into a DNA sequence. For example, if the sequence obtained in act 500 is an RNA sequence, the Us may be replaced with Ts to obtain the corresponding DNA sequence. If the sequence obtained in act 500 is a protein sequence, it may be converted into a DNA sequence using appropriate codons for the amino acids. When choosing codons for each amino acid, consideration may be given to one or more of the following factors: i) using codons that correspond to the codon bias in the organism in which the target nucleic acid may be expressed, ii) avoiding excessively high or low GC or AT contents in the target nucleic acid (for example, above 60% or below 40%; e.g., greater than 65%, 70%, 75%, 80%, 85%, or 90%; or less than 35%, 30%, 25%, 20%, 15%, or 10%), and iii) avoiding sequence features that may interfere with the assembly procedure (e.g., the presence of repeat sequences or stem loop structures). However, these factors may be ignored in some embodiments as the invention is not limited in this respect. Also, aspects of the invention may be used to reduce errors caused by one or more of these factors. Accordingly, a DNA sequence determination (e.g., a sequence determination algorithm or an automated process for determining a target DNA sequence) may omit one or more steps relating to the analysis of the GC or AT content of the target nucleic acid sequence (e.g., the GC or AT content may be ignored in some embodiments) or one or more steps relating to the analysis of certain sequence features (e.g., sequence repeats, inverted repeats, etc.) that could interfere with an assembly reaction performed under standard conditions but may not interfere with an assembly reaction including one or more concerted assembly steps.

In act 510, the sequence information may be analyzed to determine an assembly strategy. This may involve determining whether the target nucleic acid will be assembled as a single fragment or if several intermediate fragments will be assembled separately and then combined in one or more additional rounds of assembly to generate the target nucleic acid. Once the overall assembly strategy has been determined, input nucleic acids (e.g., oligonucleotides) for assembling the one or more nucleic acid fragments may be designed. The sizes and numbers of the input nucleic acids may be based in part on the type of assembly reaction (e.g., the type of polymerase-based assembly, ligase-based assembly, chemical assembly, or combination thereof) that is being used for each fragment. The input nucleic acids also may be designed to avoid 5′ and/or 3′ regions that may cross-react incorrectly and be assembled to produce undesired nucleic acid fragments. Other structural and/or sequence factors also may be considered when designing the input nucleic acids. In certain embodiments, some of the input nucleic acids may be designed to incorporate one or more specific sequences (e.g., primer binding sequences, restriction enzyme sites, etc.) at one or both ends of the assembled nucleic acid fragment.

In act 520, the input nucleic acids are obtained. These may be synthetic oligonucleotides that are synthesized on-site or obtained from a different site (e.g., from a commercial supplier). In some embodiments, one or more input nucleic acids may be amplification products (e.g., PCR products), restriction fragments, or other suitable nucleic acid molecules. Synthetic oligonucleotides may be synthesized using any appropriate technique as described in more detail herein. It should be appreciated that synthetic oligonucleotides often have sequence errors. Accordingly, oligonucleotide preparations may be selected or screened to remove error-containing molecules as described in more detail herein.

In act 530, an assembly reaction may be performed for each nucleic acid fragment. For each fragment, the input nucleic acids may be assembled using any appropriate assembly technique (e.g., a polymerase-based assembly, a ligase-based assembly, a chemical assembly, or any other multiplex nucleic acid assembly technique, or any combination thereof). An assembly reaction may result in the assembly of a number of different nucleic acid products in addition to the predetermined nucleic acid fragment. Accordingly, in some embodiments, an assembly reaction may be processed to remove incorrectly assembled nucleic acids (e.g., by size fractionation) and/or to enrich correctly assembled nucleic acids (e.g., by amplification, optionally followed by size fractionation). In some embodiments, correctly assembled nucleic acids may be amplified (e.g., in a PCR reaction) using primers that bind to the ends of the predetermined nucleic acid fragment. It should be appreciated that act 530 may be repeated one or more times. For example, in a first round of assembly a first plurality of input nucleic acids (e.g., oligonucleotides) may be assembled to generate a first nucleic acid fragment. In a second round of assembly, the first nucleic acid fragment may be combined with one or more additional nucleic acid fragments and used as starting material for the assembly of a larger nucleic acid fragment. In a third round of assembly, this larger fragment may be combined with yet further nucleic acids and used as starting material for the assembly of yet a larger nucleic acid. This procedure may be repeated as many times as needed for the synthesis of a target nucleic acid. Accordingly, progressively larger nucleic acids may be assembled. At each stage, nucleic acids of different sizes may be combined. At each stage, the nucleic acids being combined may have been previously assembled in a multiplex assembly reaction. However, at each stage, one or more nucleic acids being combined may have been obtained from different sources (e.g., PCR amplification of genomic DNA or cDNA, restriction digestion of a plasmid or genomic DNA, or any other suitable source). It should be appreciated that nucleic acids generated in each cycle of assembly may contain sequence errors if they incorporated one or more input nucleic acids with sequence error(s). Accordingly, a fidelity optimization procedure may be performed after a cycle of assembly in order to remove or correct sequence errors. It should be appreciated that fidelity optimization may be performed after each assembly reaction when several successive cycles of assembly are performed. However, in certain embodiments fidelity optimization may be performed only after a subset (e.g., 2 or more) of successive assembly reactions are complete. In some embodiments, no fidelity optimization is performed.

Accordingly, act 540 is an optional fidelity optimization procedure. Act 540 may be used in some embodiments to remove nucleic acid fragments that seem to be correctly assembled (e.g., based on their size or restriction enzyme digestion pattern) but that may have incorporated input nucleic acids containing sequence errors as described herein. For example, since synthetic oligonucleotides may contain incorrect sequences due to errors introduced during oligonucleotide synthesis, it may be useful to remove nucleic acid fragments that have incorporated one or more error-containing oligonucleotides during assembly. In some embodiments, one or more assembled nucleic acid fragments may be sequenced to determine whether they contain the predetermined sequence or not. This procedure allows fragments with the correct sequence to be identified. However, in some embodiments, other techniques may be used to remove error containing nucleic acid fragments. It should be appreciated that error containing-nucleic acids may be double-stranded homoduplexes having the error on both strands (i.e., incorrect complementary nucleotide(s), deletion(s), or addition(s) on both strands), because the assembly procedure may involve one or more rounds of polymerase extension (e.g., during assembly or after assembly to amplify the assembled product) during which an input nucleic acid containing an error may serve as a template thereby producing a complementary strand with the complementary error. In certain embodiments, a preparation of double-stranded nucleic acid fragments may be suspected to contain a mixture of nucleic acids that have the correct sequence and nucleic acids that incorporated one or more sequence errors during assembly. In some embodiments, sequence errors may be removed using a technique that involves denaturing and reannealing the double-stranded nucleic acids. In some embodiments, single strands of nucleic acids that contain complementary errors may be unlikely to reanneal together if nucleic acids containing each individual error are present in the nucleic acid preparation at a lower frequency than nucleic acids having the correct sequence at the same position. Rather, error containing single strands may reanneal with a complementary strand that contains no errors or that contains one or more different errors. As a result, error-containing strands may end up in the form of heteroduplex molecules in the reannealed reaction product. Nucleic acid strands that are error-free may reanneal with error-containing strands or with other error-free strands. Reannealed error-free strands form homoduplexes in the reannealed sample. Accordingly, by removing heteroduplex molecules from the reannealed preparation of nucleic acid fragments, the amount or frequency of error containing nucleic acids may be reduced. Any suitable method for removing heteroduplex molecules may be used, including chromatography, electrophoresis, selective binding of heteroduplex molecules, etc. In some embodiments, mismatch binding proteins that selectively (e.g., specifically) bind to heteroduplex nucleic acid molecules may be used. One example includes using MutS, a MutS homolog, or a combination thereof to bind to heteroduplex molecules. In E. coli, the MutS protein, which appears to function as a homodimer, serves as a mismatch recognition factor. In eukaryotes, at least three MutS Homolog (MSH) proteins have been identified; namely, MSH2, MSH3, and MSH6, and they form heterodimers. For example in the yeast, Saccharomyces cerevisiae, the MSH2-MSH6 complex (also known as MutSα) recognizes base mismatches and single nucleotide insertion/deletion loops, while the MSH2-MSH3 complex (also known as MutSβ) recognizes insertions/deletions of up to 12-16 nucleotides, although they exert substantially redundant functions. A mismatch binding protein may be obtained from recombinant or natural sources. A mismatch binding protein may be heat-stable. In some embodiments, a thermostable mismatch binding protein from a thermophilic organism may be used. Examples of thermostable DNA mismatch binding proteins include, but are not limited to Tth MutS (from Thermus thermophilus); Taq MutS (from Thermus aquaticus); Apy MutS (from Aquifex pyrophilus); Tma MutS (from Thermotoga maritima); any other suitable MutS; or any combination of two or more thereof.

According to aspects of the invention, protein-bound heteroduplex molecules (e.g., heteroduplex molecules bound to one or more MutS proteins) may be removed from a sample using any suitable technique (binding to a column, a filter, a nitrocellulose filter, etc., or any combination thereof). It should be appreciated that this procedure may not be 100% efficient. Some errors may remain for at least one of the following reasons. Depending on the reaction conditions, not all of the double-stranded error-containing nucleic acids may be denatured. In addition, some of the denatured error-containing strands may reanneal with complementary error-containing strands to form an error containing homoduplex. Also, the MutS/heteroduplex interaction and the MutS/heteroduplex removal procedures may not be 100% efficient. Accordingly, in some embodiments the fidelity optimization act 540 may be repeated one or more times after each assembly reaction. For example, 2, 3, 4, 5, 6, 7, 8, 9, 10 or more cycles of fidelity optimization may be performed after each assembly reaction. In some embodiments, the nucleic acid is amplified after each fidelity optimization procedure. It should be appreciated that each cycle of fidelity optimization will remove additional error-containing nucleic acid molecules. However, the proportion of correct sequences is expected to reach a saturation level after a few cycles of this procedure.

In some embodiments, the size of an assembled nucleic acid that is fidelity optimized (e.g., using MutS or a MutS homolog) may be determined by the expected number of sequence errors that are suspected to be incorporated into the nucleic acid during assembly. For example, an assembled nucleic acid product should include error free nucleic acids prior to fidelity optimization in order to be able to enrich for the error free nucleic acids. Accordingly, error screening (e.g., using MutS or a MutS homolog) should be performed on shorter nucleic acid fragments when input nucleic acids have higher error rates. In some embodiments, one or more nucleic acid fragments of between about 200 and about 800 nucleotides (e.g., about 200, about 300, about 400, about 500, about 600, about 700 or about 800 nucleotides in length) are assembled prior to fidelity optimization. After assembly, the one or more fragments may be exposed to one or more rounds of fidelity optimization as described herein. In some embodiments, several assembled fragments may be ligated together (e.g., to produce a larger nucleic acid fragment of between about 1,000 and about 5,000 bases in length, or larger), and optionally cloned into a vector, prior to fidelity optimization as described herein.

At act 550, an output nucleic acid is obtained. As discussed herein, several rounds of act 530 and/or 540 may be performed to obtain the output nucleic acid, depending on the assembly strategy that is implemented. The output nucleic acid may be amplified, cloned, stored, etc., for subsequent uses at act 560. In some embodiments, an output nucleic acid may be cloned with one or more other nucleic acids (e.g., other output nucleic acids) for subsequent applications. Subsequent applications may include one or more research, diagnostic, medical, clinical, industrial, therapeutic, environmental, agricultural, or other uses.

Aspects of the invention may include automating one or more acts described herein. For example, sequence analysis, the identification of interfering sequence features, assembly strategy selection (including fragment design and selection, etc.), fragment production, single-stranded overhang production, and/or concerted assembly may be automated in order to generate the desired product automatically. Acts of the invention may be automated using, for example, a computer system.

Aspects of the invention may be used in conjunction with any suitable multiplex nucleic acid assembly procedure. For example, concerted assembly steps may be used in connection with or more of the multiplex nucleic acid assembly procedures described below.

### Multiplex Nucleic Acid Assembly

In aspects of the invention, multiplex nucleic acid assembly relates to the assembly of a plurality of nucleic acids to generate a longer nucleic acid product. In one aspect, multiplex oligonucleotide assembly relates to the assembly of a plurality of oligonucleotides to generate a longer nucleic acid molecule. However, it should be appreciated that other nucleic acids (e.g., single or double-stranded nucleic acid degradation products, restriction fragments, amplification products, naturally occurring small nucleic acids, other polynucleotides, etc.) may be assembled or included in a multiplex assembly reaction (e.g., along with one or more oligonucleotides) in order to generate an assembled nucleic acid molecule that is longer than any of the single starting nucleic acids (e.g., oligonucleotides) that were added to the assembly reaction. In certain embodiments, one or more nucleic acid fragments that each were assembled in separate multiplex assembly reactions (e.g., separate multiplex oligonucleotide assembly reactions) may be combined and assembled to form a further nucleic acid that is longer than any of the input nucleic acid fragments. In certain embodiments, one or more nucleic acid fragments that each were assembled in separate multiplex assembly reactions (e.g., separate multiplex oligonucleotide assembly reactions) may be combined with one or more additional nucleic acids (e.g., single or double-stranded nucleic acid degradation products, restriction fragments, amplification products, naturally occurring small nucleic acids, other polynucleotides, etc.) and assembled to form a further nucleic acid that is longer than any of the input nucleic acids.

In aspects of the invention, one or more multiplex assembly reactions may be used to generate target nucleic acids having predetermined sequences. In one aspect, a target nucleic acid may have a sequence of a naturally occurring gene and/or other naturally occurring nucleic acid (e.g., a naturally occurring coding sequence, regulatory sequence, non-coding sequence, chromosomal structural sequence such as a telomere or centromere sequence, etc., any fragment thereof or any combination of two or more thereof). In another aspect, a target nucleic acid may have a sequence that is not naturally-occurring. In one embodiment, a target nucleic acid may be designed to have a sequence that differs from a natural sequence at one or more positions. In other embodiments, a target nucleic acid may be designed to have an entirely novel sequence. However, it should be appreciated that target nucleic acids may include one or more naturally occurring sequences, non-naturally occurring sequences, or combinations thereof.

In one aspect of the invention, multiplex assembly may be used to generate libraries of nucleic acids having different sequences. In some embodiments, a library may contain nucleic acids having random sequences. In certain embodiments, a predetermined target nucleic acid may be designed and assembled to include one or more random sequences at one or more predetermined positions.

In certain embodiments, a target nucleic acid may include a functional sequence (e.g., a protein binding sequence, a regulatory sequence, a sequence encoding a functional protein, etc., or any combination thereof). However, some embodiments of a target nucleic acid may lack a specific functional sequence (e.g., a target nucleic acid may include only non-functional fragments or variants of a protein binding sequence, regulatory sequence, or protein encoding sequence, or any other non-functional naturally-occurring or synthetic sequence, or any non-functional combination thereof). Certain target nucleic acids may include both functional and non-functional sequences. These and other aspects of target nucleic acids and their uses are described in more detail herein.

A target nucleic acid may be assembled in a single multiplex assembly reaction (e.g., a single oligonucleotide assembly reaction). However, a target nucleic acid also may be assembled from a plurality of nucleic acid fragments, each of which may have been generated in a separate multiplex oligonucleotide assembly reaction. It should be appreciated that one or more nucleic acid fragments generated via multiplex oligonucleotide assembly also may be combined with one or more nucleic acid molecules obtained from another source (e.g., a restriction fragment, a nucleic acid amplification product, etc.) to form a target nucleic acid. In some embodiments, a target nucleic acid that is assembled in a first reaction may be used as an input nucleic acid fragment for a subsequent assembly reaction to produce a larger target nucleic acid.

Accordingly, different strategies may be used to produce a target nucleic acid having a predetermined sequence. For example, different starting nucleic acids (e.g., different sets of predetermined nucleic acids) may be assembled to produce the same predetermined target nucleic acid sequence. Also, predetermined nucleic acid fragments may be assembled using one or more different in vitro and/or in vivo techniques. For example, nucleic acids (e.g., overlapping nucleic acid fragments) may be assembled in an in vitro reaction using an enzyme (e.g., a ligase and/or a polymerase) or a chemical reaction (e.g., a chemical ligation) or in vivo (e.g., assembled in a host cell after transfection into the host cell), or a combination thereof. Similarly, each nucleic acid fragment that is used to make a target nucleic acid may be assembled from different sets of oligonucleotides. Also, a nucleic acid fragment may be assembled using an in vitro or an in vivo technique (e.g., an in vitro or in vivo polymerase, recombinase, and/or ligase based assembly process). In addition, different in vitro assembly reactions may be used to produce a nucleic acid fragment. For example, an in vitro oligonucleotide assembly reaction may involve one or more polymerases, ligases, other suitable enzymes, chemical reactions, or any combination thereof.

### Multiplex Oligonucleotide Assembly

A predetermined nucleic acid fragment may be assembled from a plurality of different starting nucleic acids (e.g., oligonucleotides) in a multiplex assembly reaction (e.g., a multiplex enzyme-mediated reaction, a multiplex chemical assembly reaction, or a combination thereof). Certain aspects of multiplex nucleic acid assembly reactions are illustrated by the following description of certain embodiments of multiplex oligonucleotide assembly reactions. It should be appreciated that the description of the assembly reactions in the context of oligonucleotides is not intended to be limiting. The assembly reactions described herein may be performed using starting nucleic acids obtained from one or more different sources (e.g., synthetic or natural polynucleotides, nucleic acid amplification products, nucleic acid degradation products, oligonucleotides, etc.). The starting nucleic acids may be referred to as assembly nucleic acids (e.g., assembly oligonucleotides). As used herein, an assembly nucleic acid has a sequence that is designed to be incorporated into the nucleic acid product generated during the assembly process. However, it should be appreciated that the description of the assembly reactions in the context of single-stranded nucleic acids is not intended to be limiting. In some embodiments, one or more of the starting nucleic acids illustrated in the figures and described herein may be provided as double stranded nucleic acids. Accordingly, it should be appreciated that where the figures and description illustrate the assembly of single-stranded nucleic acids, the presence of one or more complementary nucleic acids is contemplated. Accordingly, one or more double-stranded complementary nucleic acids may be included in a reaction that is described herein in the context of a single-stranded assembly nucleic acid. However, in some embodiments the presence of one or more complementary nucleic acids may interfere with an assembly reaction by competing for hybridization with one of the input assembly nucleic acids. Accordingly, in some embodiments an assembly reaction may involve only single-stranded assembly nucleic acids (i.e., the assembly nucleic acids may be provided in a single-stranded form without their complementary strand) as described or illustrated herein. However, in certain embodiments the presence of one or more complementary nucleic acids may have no or little effect on the assembly reaction. In some embodiments, complementary nucleic acid(s) may be incorporated during one or more steps of an assembly. In yet further embodiments, assembly nucleic acids and their complementary strands may be assembled under the same assembly conditions via parallel assembly reactions in the same reaction mixture. In certain embodiments, a nucleic acid product resulting from the assembly of a plurality of starting nucleic acids may be identical to the nucleic acid product that results from the assembly of nucleic acids that are complementary to the starting nucleic acids (e.g., in some embodiments where the assembly steps result in the production of a double-stranded nucleic acid product). As used herein, an oligonucleotide may be a nucleic acid molecule comprising at least two covalently bonded nucleotide residues. In some embodiments, an oligonucleotide may be between 10 and 1,000 nucleotides long. For example, an oligonucleotide may be between 10 and 500 nucleotides long, or between 500 and 1,000 nucleotides long. In some embodiments, an oligonucleotide may be between about 20 and about 100 nucleotides long (e.g., from about 30 to 90, 40 to 85, 50 to 80, 60 to 75, or about 65 or about 70 nucleotides long), between about 100 and about 200, between about 200 and about 300 nucleotides, between about 300 and about 400, or between about 400 and about 500 nucleotides long. However, shorter or longer oligonucleotides may be used. An oligonucleotide may be a single-stranded nucleic acid. However, in some embodiments a double-stranded oligonucleotide may be used as described herein. In certain embodiments, an oligonucleotide may be chemically synthesized as described in more detail below.

In some embodiments, an input nucleic acid (e.g., oligonucleotide) may be amplified before use. The resulting product may be double-stranded. In some embodiments, one of the strands of a double-stranded nucleic acid may be removed before use so that only a predetermined single strand is added to an assembly reaction.

In certain embodiments, each oligonucleotide may be designed to have a sequence that is identical to a different portion of the sequence of a predetermined target nucleic acid that is to be assembled. Accordingly, in some embodiments each oligonucleotide may have a sequence that is identical to a portion of one of the two strands of a double-stranded target nucleic acid. For clarity, the two complementary strands of a double stranded nucleic acid are referred to herein as the positive (P) and negative (N) strands. This designation is not intended to imply that the strands are sense and anti-sense strands of a coding sequence. They refer only to the two complementary strands of a nucleic acid (e.g., a target nucleic acid, an intermediate nucleic acid fragment, etc.) regardless of the sequence or function of the nucleic acid. Accordingly, in some embodiments a P strand may be a sense strand of a coding sequence, whereas in other embodiments a P strand may be an anti-sense strand of a coding sequence. According to the invention, a target nucleic acid may be either the P strand, the N strand, or a double-stranded nucleic acid comprising both the P and N strands.

It should be appreciated that different oligonucleotides may be designed to have different lengths. In some embodiments, one or more different oligonucleotides may have overlapping sequence regions (e.g., overlapping 5′ regions or overlapping 3′ regions). Overlapping sequence regions may be identical (i.e., corresponding to the same strand of the nucleic acid fragment) or complementary (i.e., corresponding to complementary strands of the nucleic acid fragment). The plurality of oligonucleotides may include one or more oligonucleotide pairs with overlapping identical sequence regions, one or more oligonucleotide pairs with overlapping complementary sequence regions, or a combination thereof. Overlapping sequences may be of any suitable length. For example, overlapping sequences may encompass the entire length of one or more nucleic acids used in an assembly reaction. Overlapping sequences may be between about 5 and about 500 nucleotides long (e.g., between about 10 and 100, between about 10 and 75, between about 10 and 50, about 20, about 25, about 30, about 35, about 40, about 45, about 50, etc.) However, shorter, longer or intermediate overlapping lengths may be used. It should be appreciated that overlaps between different input nucleic acids used in an assembly reaction may have different lengths.

In a multiplex oligonucleotide assembly reaction designed to generate a predetermined nucleic acid fragment, the combined sequences of the different oligonucleotides in the reaction may span the sequence of the entire nucleic acid fragment on either the positive strand, the negative strand, both strands, or a combination of portions of the positive strand and portions of the negative strand. The plurality of different oligonucleotides may provide either positive sequences, negative sequences, or a combination of both positive and negative sequences corresponding to the entire sequence of the nucleic acid fragment to be assembled. In some embodiments, the plurality of oligonucleotides may include one or more oligonucleotides having sequences identical to one or more portions of the positive sequence, and one or more oligonucleotides having sequences that are identical to one or more portions of the negative sequence of the nucleic acid fragment. One or more pairs of different oligonucleotides may include sequences that are identical to overlapping portions of the predetermined nucleic acid fragment sequence as described herein (e.g., overlapping sequence portions from the same or from complementary strands of the nucleic acid fragment). In some embodiments, the plurality of oligonucleotides includes a set of oligonucleotides having sequences that combine to span the entire positive sequence and a set oligonucleotides having sequences that combine to span the entire negative sequence of the predetermined nucleic acid fragment. However, in certain embodiments, the plurality of oligonucleotides may include one or more oligonucleotides with sequences that are identical to sequence portions on one strand (either the positive or negative strand) of the nucleic acid fragment, but no oligonucleotides with sequences that are complementary to those sequence portions. In one embodiment, a plurality of oligonucleotides includes only oligonucleotides having sequences identical to portions of the positive sequence of the predetermined nucleic acid fragment. In one embodiment, a plurality of oligonucleotides includes only oligonucleotides having sequences identical to portions of the negative sequence of the predetermined nucleic acid fragment. These oligonucleotides may be assembled by sequential ligation or in an extension-based reaction (e.g., if an oligonucleotide having a 3′ region that is complementary to one of the plurality of oligonucleotides is added to the reaction).

In one aspect, a nucleic acid fragment may be assembled in a polymerase-mediated assembly reaction from a plurality of oligonucleotides that are combined and extended in one or more rounds of polymerase-mediated extensions. In another aspect, a nucleic acid fragment may be assembled in a ligase-mediated reaction from a plurality of oligonucleotides that are combined and ligated in one or more rounds of ligase-mediated ligations. In another aspect, a nucleic acid fragment may be assembled in a non-enzymatic reaction (e.g., a chemical reaction) from a plurality of oligonucleotides that are combined and assembled in one or more rounds of non-enzymatic reactions. In some embodiments, a nucleic acid fragment may be assembled using a combination of polymerase, ligase, and/or non-enzymatic reactions. For example, both polymerase(s) and ligase(s) may be included in an assembly reaction mixture. Accordingly, a nucleic acid may be assembled via coupled amplification and ligation or ligation during amplification. The resulting nucleic acid fragment from each assembly technique may have a sequence that includes the sequences of each of the plurality of assembly oligonucleotides that were used as described herein. These assembly reactions may be referred to as primerless assemblies, since the target nucleic acid is generated by assembling the input oligonucleotides rather than being generated in an amplification reaction where the oligonucleotides act as amplification primers to amplify a pre-existing template nucleic acid molecule corresponding to the target nucleic acid.

Polymerase-based assembly techniques may involve one or more suitable polymerase enzymes that can catalyze a template-based extension of a nucleic acid in a 5′ to 3′ direction in the presence of suitable nucleotides and an annealed template. A polymerase may be thermostable. A polymerase may be obtained from recombinant or natural sources. In some embodiments, a thermostable polymerase from a thermophilic organism may be used. In some embodiments, a polymerase may include a 3′→5′ exonuclease/proofreading activity. In some embodiments, a polymerase may have no, or little, proofreading activity (e.g., a polymerase may be a recombinant variant of a natural polymerase that has been modified to reduce its proofreading activity). Examples of thermostable DNA polymerases include, but are not limited to: Taq (a heat-stable DNA polymerase from the bacterium Thermus aquaticus); Pfu (a thermophilic DNA polymerase with a 3′→5′ exonuclease/proofreading activity from Pyrococcus furiosus, available from for example Promega); VentR® DNA Polymerase and VentR® (exo-) DNA Polymerase (thermophilic DNA polymerases with or without a 3′→5′ exonuclease/proofreading activity from Thermococcus litoralis; also known as Tli polymerase); Deep VentR® DNA Polymerase and Deep VentR® (exo-) DNA Polymerase (thermophilic DNA polymerases with or without a 3′→5′ exonuclease/proofreading activity from Pyrococcus species GB-D; available from New England Biolabs); KOD HiFi (a recombinant Thermococcus kodakaraensis KOD1 DNA polymerase with a 3′→5′ exonuclease/proofreading activity, available from Novagen,); BIO-X-ACT (a mix of polymerases that possesses 5′→3′ DNA polymerase activity and 3′→5′ proofreading activity); Klenow Fragment (an N-terminal truncation of E. coli DNA Polymerase I which retains polymerase activity, but has lost the 5′→3′ exonuclease activity, available from, for example, Promega and NEB); Sequenase™ (T7 DNA polymerase deficient in 3′-5′ exonuclease activity); Phi29 (bacteriophage 29 DNA polymerase, may be used for rolling circle amplification, for example, in a TempliPhi™ DNA Sequencing Template Amplification Kit, available from Amersham Biosciences); TopoTaq™ (a hybrid polymerase that combines hyperstable DNA binding domains and the DNA unlinking activity of Methanopyrus topoisomerase, with no exonuclease activity, available from Fidelity Systems); TopoTaq HiFi which incorporates a proofreading domain with exonuclease activity; Phusion™ (a Pyrococcus-like enzyme with a processivity-enhancing domain, available from New England Biolabs); any other suitable DNA polymerase, or any combination of two or more thereof.

Ligase-based assembly techniques may involve one or more suitable ligase enzymes that can catalyze the covalent linking of adjacent 3′ and 5′ nucleic acid termini (e.g., a 5′ phosphate and a 3′ hydroxyl of nucleic acid(s) annealed on a complementary template nucleic acid such that the 3′ terminus is immediately adjacent to the 5′ terminus). Accordingly, a ligase may catalyze a ligation reaction between the 5′ phosphate of a first nucleic acid to the 3′ hydroxyl of a second nucleic acid if the first and second nucleic acids are annealed next to each other on a template nucleic acid). A ligase may be obtained from recombinant or natural sources. A ligase may be a heat-stable ligase. In some embodiments, a thermostable ligase from a thermophilic organism may be used. Examples of thermostable DNA ligases include, but are not limited to: Tth DNA ligase (from Thermus thermophilus, available from, for example, Eurogentec and GeneCraft); Pfu DNA ligase (a hyperthermophilic ligase from Pyrococcus furiosus); Taq ligase (from Thermus aquaticus), any other suitable heat-stable ligase, or any combination thereof. In some embodiments, one or more lower temperature ligases may be used (e.g., T4 DNA ligase). A lower temperature ligase may be useful for shorter overhangs (e.g., about 3, about 4, about 5, or about 6 base overhangs) that may not be stable at higher temperatures.

Non-enzymatic techniques can be used to ligate nucleic acids. For example, a 5′-end (e.g., the 5′ phosphate group) and a 3′-end (e.g., the 3′ hydroxyl) of one or more nucleic acids may be covalently linked together without using enzymes (e.g., without using a ligase). In some embodiments, non-enzymatic techniques may offer certain advantages over enzyme-based ligations. For example, non-enzymatic techniques may have a high tolerance of non-natural nucleotide analogues in nucleic acid substrates, may be used to ligate short nucleic acid substrates, may be used to ligate RNA substrates, and/or may be cheaper and/or more suited to certain automated (e.g., high throughput) applications.

Non-enzymatic ligation may involve a chemical ligation. In some embodiments, nucleic acid termini of two or more different nucleic acids may be chemically ligated. In some embodiments, nucleic acid termini of a single nucleic acid may be chemically ligated (e.g., to circularize the nucleic acid). It should be appreciated that both strands at a first double-stranded nucleic acid terminus may be chemically ligated to both strands at a second double-stranded nucleic acid terminus. However, in some embodiments only one strand of a first nucleic acid terminus may be chemically ligated to a single strand of a second nucleic acid terminus. For example, the 5′ end of one strand of a first nucleic acid terminus may be ligated to the 3′ end of one strand of a second nucleic acid terminus without the ends of the complementary strands being chemically ligated.

Accordingly, a chemical ligation may be used to form a covalent linkage between a 5′ terminus of a first nucleic acid end and a 3′ terminus of a second nucleic acid end, wherein the first and second nucleic acid ends may be ends of a single nucleic acid or ends of separate nucleic acids. In one aspect, chemical ligation may involve at least one nucleic acid substrate having a modified end (e.g., a modified 5′ and/or 3′ terminus) including one or more chemically reactive moieties that facilitate or promote linkage formation. In some embodiments, chemical ligation occurs when one or more nucleic acid termini are brought together in close proximity (e.g., when the termini are brought together due to annealing between complementary nucleic acid sequences). Accordingly, annealing between complementary 3′ or 5′ overhangs (e.g., overhangs generated by restriction enzyme cleavage of a double-stranded nucleic acid) or between any combination of complementary nucleic acids that results in a 3′ terminus being brought into close proximity with a 5′ terminus (e.g., the 3′ and 5′ termini are adjacent to each other when the nucleic acids are annealed to a complementary template nucleic acid) may promote a template-directed chemical ligation. Examples of chemical reactions may include, but are not limited to, condensation, reduction, and/or photo-chemical ligation reactions. It should be appreciated that in some embodiments chemical ligation can be used to produce naturally-occurring phosphodiester internucleotide linkages, non-naturally-occurring phosphamide pyrophosphate internucleotide linkages, and/or other non-naturally-occurring internucleotide linkages.

In some embodiments, the process of chemical ligation may involve one or more coupling agents to catalyze the ligation reaction. A coupling agent may promote a ligation reaction between reactive groups in adjacent nucleic acids (e.g., between a 5′-reactive moiety and a 3′-reactive moiety at adjacent sites along a complementary template). In some embodiments, a coupling agent may be a reducing reagent (e.g., ferricyanide), a condensing reagent such (e.g., cyanoimidazole, cyanogen bromide, carbodiimide, etc.), or irradiation (e.g., UV irradiation for photo-ligation).

In some embodiments, a chemical ligation may be an autoligation reaction that does not involve a separate coupling agent. In autoligation, the presence of a reactive group on one or more nucleic acids may be sufficient to catalyze a chemical ligation between nucleic acid termini without the addition of a coupling agent (see, for example, Xu Y & Kool E T, 1997, Tetrahedron Lett. 38:5595-8). Non-limiting examples of these reagent-free ligation reactions may involve nucleophilic displacements of sulfur on bromoacetyl, tosyl, or iodo-nucleoside groups (see, for example, Xu Y et al., 2001, Nat Biotech 19:148-52). Nucleic acids containing reactive groups suitable for autoligation can be prepared directly on automated synthesizers (see, for example, Xu Y & Kool E T, 1999, Nuc. Acids Res. 27:875-81). In some embodiments, a phosphorothioate at a 3′ terminus may react with a leaving group (such as tosylate or iodide) on a thymidine at an adjacent 5′ terminus. In some embodiments, two nucleic acid strands bound at adjacent sites on a complementary target strand may undergo auto-ligation by displacement of a 5′-end iodide moiety (or tosylate) with a 3′-end sulfur moiety. Accordingly, in some embodiments the product of an autoligation may include a non-naturally-occurring internucleotide linkage (e.g., a single oxygen atom may be replaced with a sulfur atom in the ligated product).

In some embodiments, a synthetic nucleic acid duplex can be assembled via chemical ligation in a one step reaction involving simultaneous chemical ligation of nucleic acids on both strands of the duplex. For example, a mixture of 5′-phosphorylated oligonucleotides corresponding to both strands of a target nucleic acid may be chemically ligated by a) exposure to heat (e.g., to 97° C.) and slow cooling to form a complex of annealed oligonucleotides, and b) exposure to cyanogen bromide or any other suitable coupling agent under conditions sufficient to chemically ligate adjacent 3′ and 5′ ends in the nucleic acid complex.

In some embodiments, a synthetic nucleic acid duplex can be assembled via chemical ligation in a two step reaction involving separate chemical ligations for the complementary strands of the duplex. For example, each strand of a target nucleic acid may be ligated in a separate reaction containing phosphorylated oligonucleotides corresponding to the strand that is to be ligated and non-phosphorylated oligonucleotides corresponding to the complementary strand. The non-phosphorylated oligonucleotides may serve as a template for the phosphorylated oligonucleotides during a chemical ligation (e.g. using cyanogen bromide). The resulting single-stranded ligated nucleic acid may be purified and annealed to a complementary ligated single-stranded nucleic acid to form the target duplex nucleic acid (see, for example, Shabarova Z A et al., 1991, Nuc. Acids Res. 19:4247-51).

Aspects of the invention may be used to enhance different types of nucleic acid assembly reactions (e.g., multiplex nucleic acid assembly reactions). Aspects of the invention may be used in combination with one or more assembly reactions described in, for example, Carr et al., 2004, Nucleic Acids Research, Vol. 32, No 20, e162 (9 pages); Richmond et al., 2004, Nucleic Acids Research, Vol. 32, No 17, pp. 5011-5018; Caruthers et al., 1972, J. Mol. Biol. 72, 475-492; Hecker et al., 1998, Biotechniques 24:256-260; Kodumal et al., 2004, PNAS Vol. 101, No. 44, pp. 15573-15578; Tian et al., 2004, Nature, Vol. 432, pp. 1050-1054; and U.S. Pat. Nos. 6,008,031 and 5,922,539, the disclosures of which are incorporated herein by reference. Certain embodiments of multiplex nucleic acid assembly reactions for generating a predetermined nucleic acid fragment are illustrated with reference to FIGS. 1-4. It should be appreciated that synthesis and assembly methods described herein (including, for example, oligonucleotide synthesis, multiplex nucleic acid assembly, concerted assembly of nucleic acid fragments, or any combination thereof) may be performed in any suitable format, including in a reaction tube, in a multi-well plate, on a surface, on a column, in a microfluidic device (e.g., a microfluidic tube), a capillary tube, etc. It should be appreciated that the reference to complementary nucleic acids or complementary nucleic acid regions herein refers to nucleic acids or regions thereof that have sequences which are reverse complements of each other so that they can hybridize in an antiparallel fashion typical of natural DNA.

FIG. 1 shows one embodiment of a plurality of oligonucleotides that may be assembled in a polymerase-based multiplex oligonucleotide assembly reaction. FIG. 1A shows two groups of oligonucleotides (Group P and Group N) that have sequences of portions of the two complementary strands of a nucleic acid fragment to be assembled. Group P includes oligonucleotides with positive strand sequences (P₁, P₂, . . . Pₙ₋₁, Pₙ, Pₙ₊₁, . . . P_{T}, shown from 5′→3′ on the positive strand). Group N includes oligonucleotides with negative strand sequences (N_{T}, . . . , Nₙ₊₁, Nₙ, Nₙ₋₁, . . . , N₂, N₁, shown from 5′→3′ on the negative strand). In this example, none of the P group oligonucleotides overlap with each other and none of the N group oligonucleotides overlap with each other. However, in some embodiments, one or more of the oligonucleotides within the S or N group may overlap. Furthermore, FIG. 1A shows gaps between consecutive oligonucleotides in Group P and gaps between consecutive oligonucleotides in Group N. However, each P group oligonucleotide (except for P₁) and each N group oligonucleotide (except for N_{T}) overlaps with complementary regions of two oligonucleotides from the complementary group of oligonucleotides. P₁ and N_{T} overlap with a complementary region of only one oligonucleotide from the other group (the complementary 3′-most oligonucleotides N₁ and P_{T}, respectively). FIG. 1B shows a structure of an embodiment of a Group P or Group N oligonucleotide represented in FIG. 1A. This oligonucleotide includes a 5′ region that is complementary to a 5′ region of a first oligonucleotide from the other group, a 3′ region that is complementary to a 3′ region of a second oligonucleotide from the other group, and a core or central region that is not complementary to any oligonucleotide sequence from the other group (or its own group). This central region is illustrated as the B region in FIG. 1B. The sequence of the B region may be different for each different oligonucleotide. As defined herein, the B region of an oligonucleotide in one group corresponds to a gap between two consecutive oligonucleotides in the complementary group of oligonucleotides. It should be noted that the 5′-most oligonucleotide in each group (P₁ in Group P and N_{T} in Group N) does not have a 5′ region that is complementary to the 5′ region of any other oligonucleotide in either group. Accordingly, the 5′-most oligonucleotides (P₁ and N_{T}) that are illustrated in FIG. 1A each have a 3′ complementary region and a 5′ non-complementary region (the B region of FIG. 1B), but no 5′ complementary region. However, it should be appreciated that any one or more of the oligonucleotides in Group P and/or Group N (including all of the oligonucleotides in Group P and/or Group N) can be designed to have no B region. In the absence of a B region, a 5′-most oligonucleotide has only the 3′ complementary region (meaning that the entire oligonucleotide is complementary to the 3′ region of the 3′-most oligonucleotide from the other group (e.g., the 3′ region of N₁ or P_{T} shown in FIG. 1A). In the absence of a B region, one of the other oligonucleotides in either Group P or Group N has only a 5′ complementary region and a 3′ complementary region (meaning that the entire oligonucleotide is complementary to the 5′ and 3′ sequence regions of the two overlapping oligonucleotides from the complementary group). In some embodiments, only a subset of oligonucleotides in an assembly reaction may include B regions. It should be appreciated that the length of the 5′, 3′, and B regions may be different for each oligonucleotide. However, for each oligonucleotide the length of the 5′ region is the same as the length of the complementary 5′ region in the 5′ overlapping oligonucleotide from the other group. Similarly, the length of the 3′ region is the same as the length of the complementary 3′ region in the 3′ overlapping oligonucleotide from the other group. However, in certain embodiments a 3′-most oligonucleotide may be designed with a 3′ region that extends beyond the 5′ region of the 5′-most oligonucleotide. In this embodiment, an assembled product may include the 5′ end of the 5′-most oligonucleotide, but not the 3′ end of the 3′-most oligonucleotide that extends beyond it.

FIG. 1C illustrates a subset of the oligonucleotides from FIG. 1A, each oligonucleotide having a 5′, a 3′, and an optional B region. Oligonucleotide Pₙ is shown with a 5′ region that is complementary to (and can anneal to) the 5′ region of oligonucleotide Nₙ₋₁. Oligonucleotide Pₙ also has a 3′ region that is complementary to (and can anneal to) the 3′ region of oligonucleotide Nₙ. Nₙ is also shown with a 5′ region that is complementary (and can anneal to) the 5′ region of oligonucleotide Pₙ₊₁. This pattern could be repeated for all of oligonucleotides P₂ to P_{T} and N₁ to N_{T-1} (with the 5′-most oligonucleotides only having 3′ complementary regions as discussed herein). If all of the oligonucleotides from Group P and Group N are mixed together under appropriate hybridization conditions, they may anneal to form a long chain such as the oligonucleotide complex illustrated in FIG. 1A. However, subsets of the oligonucleotides may form shorter chains and even oligonucleotide dimers with annealed 5′ or 3′ regions. It should be appreciated that many copies of each oligonucleotide are included in a typical reaction mixture. Accordingly, the resulting hybridized reaction mixture may contain a distribution of different oligonucleotide dimers and complexes. Polymerase-mediated extension of the hybridized oligonucleotides results in a template-based extension of the 3′ ends of oligonucleotides that have annealed 3′ regions. Accordingly, polymerase-mediated extension of the oligonucleotides shown in FIG. 1C would result in extension of the 3′ ends only of oligonucleotides Pₙ and Nₙ generating extended oligonucleotides containing sequences that are complementary to all the regions of Nₙ and Pₙ, respectively. Extended oligonucleotide products with sequences complementary to all of Nₙ₋₁ and Pₙ₊₁ would not be generated unless oligonucleotides Pₙ₋₁ and Nₙ₊₁ were included in the reaction mixture. Accordingly, if all of the oligonucleotide sequences in a plurality of oligonucleotides are to be incorporated into an assembled nucleic acid fragment using a polymerase, the plurality of oligonucleotides should include 5′-most oligonucleotides that are at least complementary to the entire 3′ regions of the 3′-most oligonucleotides. In some embodiments, the 5′-most oligonucleotides also may have 5′ regions that extend beyond the 3′ ends of the 3′-most oligonucleotides as illustrated in FIG. 1A. In some embodiments, a ligase also may be added to ligate adjacent 5′ and 3′ ends that may be formed upon 3′ extension of annealed oligonucleotides in an oligonucleotide complex such as the one illustrated in FIG. 1A.

When assembling a nucleic acid fragment using a polymerase, a single cycle of polymerase extension extends oligonucleotide pairs with annealed 3′ regions. Accordingly, if a plurality of oligonucleotides were annealed to form an annealed complex such as the one illustrated in FIG. 1A, a single cycle of polymerase extension would result in the extension of the 3′ ends of the P₁/N₁, P₂/N₂, . . . , P₁/N₁, P₁/N₁, Pₙ₊₁/N₊₁, . . . , P_{T}/N_{T} oligonucleotide pairs. In one embodiment, a single molecule could be generated by ligating the extended oligonucleotide dimers. In one embodiment, a single molecule incorporating all of the oligonucleotide sequences may be generated by performing several polymerase extension cycles.

In one embodiment, FIG. 1D illustrates two cycles of polymerase extension (separated by a denaturing step and an annealing step) and the resulting nucleic acid products. It should be appreciated that several cycles of polymerase extension may be required to assemble a single nucleic acid fragment containing all the sequences of an initial plurality of oligonucleotides. In one embodiment, a minimal number of extension cycles for assembling a nucleic acid may be calculated as log₂n, where n is the number of oligonucleotides being assembled. In some embodiments, progressive assembly of the nucleic acid may be achieved without using temperature cycles. For example, an enzyme capable of rolling circle amplification may be used (e.g., phi 29 polymerase) when a circularized nucleic acid (e.g., oligonucleotide) complex is used as a template to produce a large amount of circular product for subsequent processing using MutS or a MutS homolog as described herein. In step 1 of FIG. 1D, annealed oligonucleotide pairs Pₙ/Nₙ and Pₙ₊₁/Nₙ₊₁ are extended to form oligonucleotide dimer products incorporating the sequences covered by the respective oligonucleotide pairs. For example, Pₙ is extended to incorporate sequences that are complementary to the B and 5′ regions of Nₙ (indicated as N′ₙ in FIG. 1D). Similarly, Nₙ₊₁ is extended to incorporate sequences that are complementary to the 5′ and B regions of Pₙ₊₁ (indicated as Pₙ₊₁ in FIG. 1D). These dimer products may be denatured and reannealed to form the starting material of step 2 where the 3′ end of the extended Pₙ oligonucleotide is annealed to the 3′ end of the extended Nₙ₊₁ oligonucleotide. This product may be extended in a polymerase-mediated reaction to form a product that incorporates the sequences of the four oligonucleotides (Pₙ, Nₙ, Pₙ+1, Nₙ₊₁). One strand of this extended product has a sequence that includes (in 5′ to 3′ order) the 5′, B, and 3′ regions of Pₙ, the complement of the B region of Nₙ, the 5′, B, and 3′ regions of Pₙ₊₁, and the complements of the B and 5′ regions of Nₙ₊₁. The other strand of this extended product has the complementary sequence. It should be appreciated that the 3′ regions of Pₙ and Nₙ are complementary, the 5′ regions of Nₙ and P₊₁ are complementary, and the 3′ regions of Pₙ₊₁ and Nₙ₊₁ are complementary. It also should be appreciated that the reaction products shown in FIG. 1D are a subset of the reaction products that would be obtained using all of the oligonucleotides of Group P and Group N. A first polymerase extension reaction using all of the oligonucleotides would result in a plurality of overlapping oligonucleotide dimers from P₁/N₁ to P_{T}/N_{T}. Each of these may be denatured and at least one of the strands could then anneal to an overlapping complementary strand from an adjacent (either 3′ or 5′) oligonucleotide dimer and be extended in a second cycle of polymerase extension as shown in FIG. 1D. Subsequent cycles of denaturing, annealing, and extension produce progressively larger products including a nucleic acid fragment that includes the sequences of all of the initial oligonucleotides. It should be appreciated that these subsequent rounds of extension also produce many nucleic acid products of intermediate length. The reaction product may be complex since not all of the 3′ regions may be extended in each cycle. Accordingly, unextended oligonucleotides may be available in each cycle to anneal to other unextended oligonucleotides or to previously extended oligonucleotides. Similarly, extended products of different sizes may anneal to each other in each cycle. Accordingly, a mixture of extended products of different sizes covering different regions of the sequence may be generated along with the nucleic acid fragment covering the entire sequence. This mixture also may contain any remaining unextended oligonucleotides.

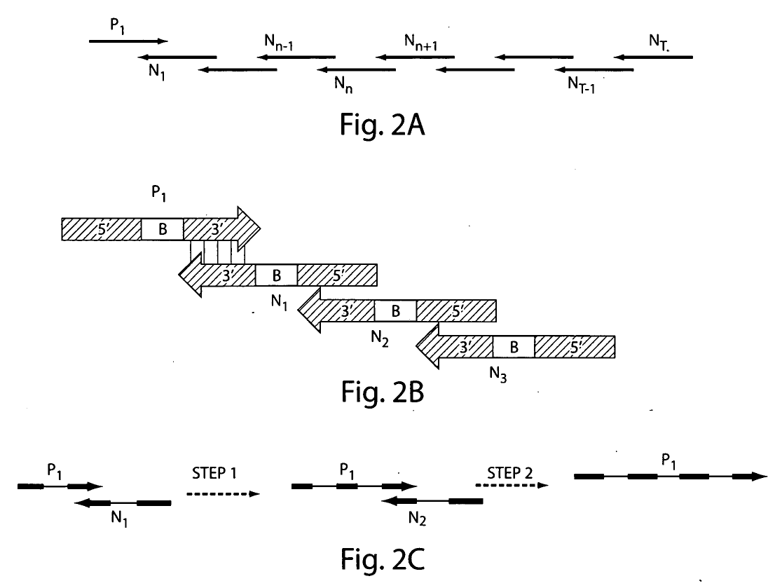

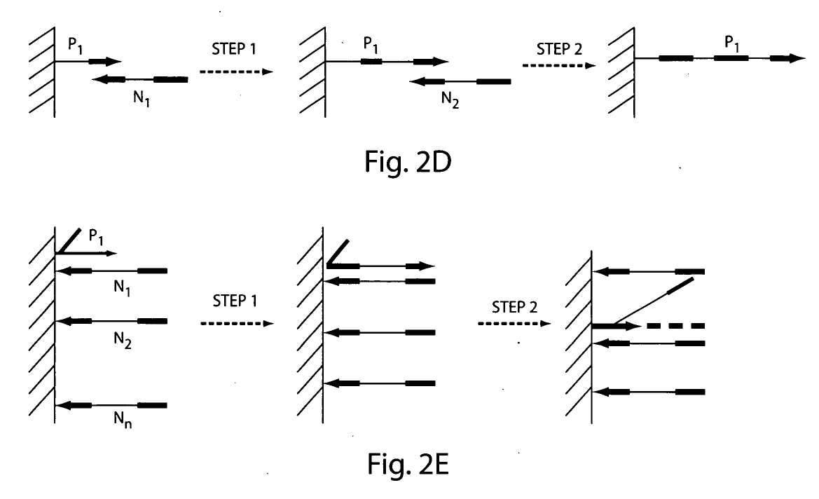

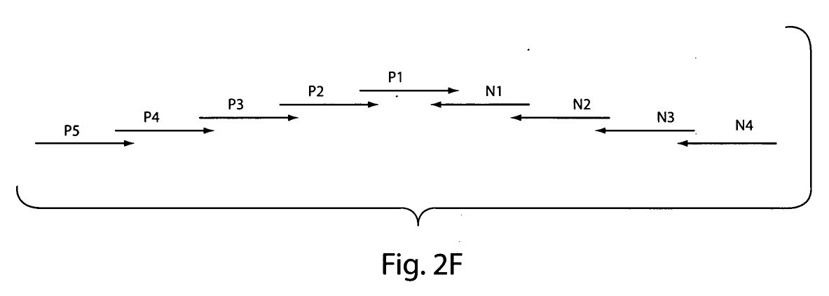

FIG. 2 shows an embodiment of a plurality of oligonucleotides that may be assembled in a directional polymerase-based multiplex oligonucleotide assembly reaction. In this embodiment, only the 5′-most oligonucleotide of Group P may be provided. In contrast to the example shown in FIG. 1, the remainder of the sequence of the predetermined nucleic acid fragment is provided by oligonucleotides of Group N. The 3′-most oligonucleotide of Group N (N1) has a 3′ region that is complementary to the 3′ region of P₁ as shown in FIG. 2B. However, the remainder of the oligonucleotides in Group N have overlapping (but non-complementary) 3′ and 5′ regions as illustrated in FIG. 2B for oligonucleotides N1-N3. Each Group N oligonucleotide (e.g., Nₙ) overlaps with two adjacent oligonucleotides: one overlaps with the 3′ region (Nₙ₋₁) and one with the 5′ region (Nₙ₊₁), except for N₁ that overlaps with the 3′ regions of P₁ (complementary overlap) and N2 (non-complementary overlap), and NT that overlaps only with N_{T-1}. It should be appreciated that all of the overlaps shown in FIG. 2A between adjacent oligonucleotides N₂ to N_{T-1} are non-complementary overlaps between the 5′ region of one oligonucleotide and the 3′ region of the adjacent oligonucleotide illustrated in a 3′ to 5′ direction on the N strand of the predetermined nucleic acid fragment. It also should be appreciated that each oligonucleotide may have 3′, B, and 5′ regions of different lengths (including no B region in some embodiments). In some embodiments, none of the oligonucleotides may have B regions, meaning that the entire sequence of each oligonucleotide may overlap with the combined 5′ and 3′ region sequences of its two adjacent oligonucleotides.

Assembly of a predetermined nucleic acid fragment from the plurality of oligonucleotides shown in FIG. 2A may involve multiple cycles of polymerase-mediated extension. Each extension cycle may be separated by a denaturing and an annealing step.

FIG. 2C illustrates the first two steps in this assembly process. In step 1, annealed oligonucleotides P₁ and N₁ are extended to form an oligonucleotide dimer. P₁ is shown with a 5′ region that is non-complementary to the 3′ region of N₁ and extends beyond the 3′ region of N₁ when the oligonucleotides are annealed. However, in some embodiments, P₁ may lack the 5′ non-complementary region and include only sequences that overlap with the 3′ region of N₁. The product of P₁ extension is shown after step 1 containing an extended region that is complementary to the 5′ end of N₁. The single strand illustrated in FIG. 2C may be obtained by denaturing the oligonucleotide dimer that results from the extension of P₁/N₁ in step 1. The product of P₁ extension is shown annealed to the 3′ region of N₂. This annealed complex may be extended in step 2 to generate an extended product that now includes sequences complementary to the B and 5′ regions of N₂. Again, the single strand illustrated in FIG. 2C may be obtained by denaturing the oligonucleotide dimer that results from the extension reaction of step 2. Additional cycles of extension may be performed to further assemble a predetermined nucleic acid fragment. In each cycle, extension results in the addition of sequences complementary to the B and 5′ regions of the next Group N oligonucleotide. Each cycle may include a denaturing and annealing step. However, the extension may occur under the annealing conditions. Accordingly, in one embodiment, cycles of extension may be obtained by alternating between denaturing conditions (e.g., a denaturing temperature) and annealing/extension conditions (e.g., an annealing/extension temperature). In one embodiment, T (the number of group N oligonucleotides) may determine the minimal number of temperature cycles used to assemble the oligonucleotides. However, in some embodiments, progressive extension may be achieved without temperature cycling. For example, an enzyme capable promoting rolling circle amplification may be used (e.g., TempliPhi). It should be appreciated that a reaction mixture containing an assembled predetermined nucleic acid fragment also may contain a distribution of shorter extension products that may result from incomplete extension during one or more of the cycles or may be the result of an P₁/N₁ extension that was initiated after the first cycle.

FIG. 2D illustrates an example of a sequential extension reaction where the 5′-most P₁ oligonucleotide is bound to a support and the Group N oligonucleotides are unbound. The reaction steps are similar to those described for FIG. 2C. However, an extended predetermined nucleic acid fragment will be bound to the support via the 5′-most P₁ oligonucleotide. Accordingly, the complementary strand (the negative strand) may readily be obtained by denaturing the bound fragment and releasing the negative strand. In some embodiments, the attachment to the support may be labile or readily reversed (e.g., using light, a chemical reagent, a pH change, etc.) and the positive strand also may be released. Accordingly, either the positive strand, the negative strand, or the double-stranded product may be obtained. FIG. 2E illustrates an example of a sequential reaction where P₁ is unbound and the Group N oligonucleotides are bound to a support. The reaction steps are similar to those described for FIG. 2C. However, an extended predetermined nucleic acid fragment will be bound to the support via the 5′-most N_{T} oligonucleotide. Accordingly, the complementary strand (the positive strand) may readily be obtained by denaturing the bound fragment and releasing the positive strand. In some embodiments, the attachment to the support may be labile or readily reversed (e.g., using light, a chemical reagent, a pH change, etc.) and the negative strand also may be released. Accordingly, either the positive strand, the negative strand, or the double-stranded product may be obtained.

It should be appreciated that other configurations of oligonucleotides may be used to assemble a nucleic acid via two or more cycles of polymerase-based extension. In many configurations, at least one pair of oligonucleotides have complementary 3′ end regions. FIG. 2F illustrates an example where an oligonucleotide pair with complementary 3′ end regions is flanked on either side by a series of oligonucleotides with overlapping non-complementary sequences. The oligonucleotides illustrated to the right of the complementary pair have overlapping 3′ and 5′ regions (with the 3′ region of one oligonucleotide being identical to the 5′ region of the adjacent oligonucleotide) that corresponding to a sequence of one strand of the target nucleic acid to be assembled. The oligonucleotides illustrated to the left of the complementary pair have overlapping 3′ and 5′ regions (with the 3′ region of one oligonucleotide being identical to the 5′ region of the adjacent oligonucleotide) that correspond to a sequence of the complementary strand of the target nucleic acid. These oligonucleotides may be assembled via sequential polymerase-based extension reactions as described herein (see also, for example, Xiong et al., 2004, Nucleic Acids Research, Vol. 32, No. 12, e98, 10 pages, the disclosure of which is incorporated by reference herein). It should be appreciated that different numbers and/or lengths of oligonucleotides may be used on either side of the complementary pair. Accordingly, the illustration of the complementary pair as the central pair in FIG. 2F is not intended to be limiting as other configuration of a complementary oligonucleotide pair flanked by a different number of non-complementary pairs on either side may be used according to methods of the invention.

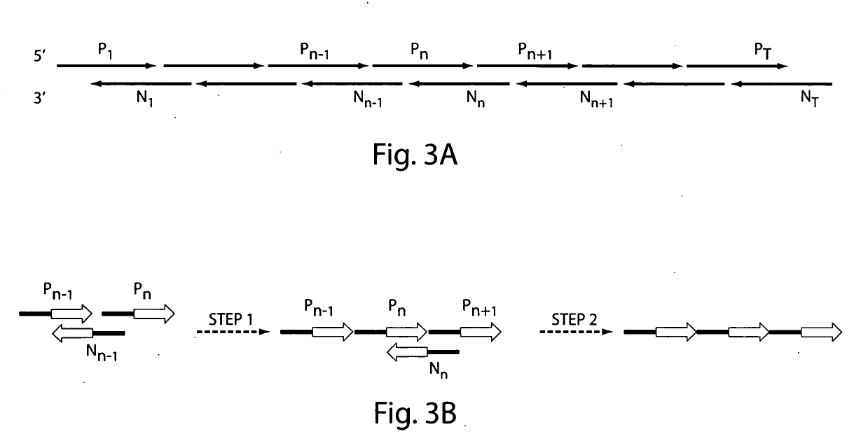

FIG. 3 shows an embodiment of a plurality of oligonucleotides that may be assembled in a ligase reaction. FIG. 3A illustrates the alignment of the oligonucleotides showing that they do not contain gaps (i.e., no B region as described herein). Accordingly, the oligonucleotides may anneal to form a complex with no nucleotide gaps between the 3′ and 5′ ends of the annealed oligonucleotides in either Group P or Group N. These oligonucleotides provide a suitable template for assembly using a ligase under appropriate reaction conditions. However, it should be appreciated that these oligonucleotides also may be assembled using a polymerase-based assembly reaction as described herein. FIG. 3B shows two individual ligation reactions. These reactions are illustrated in two steps. However, it should be appreciated that these ligation reactions may occur simultaneously or sequentially in any order and may occur as such in a reaction maintained under constant reaction conditions (e.g., with no temperature cycling) or in a reaction exposed to several temperature cycles. For example, the reaction illustrated in step 2 may occur before the reaction illustrated in step 1. In each ligation reaction illustrated in FIG. 3B, a Group N oligonucleotide is annealed to two adjacent Group P oligonucleotides (due to the complementary 5′ and 3′ regions between the P and N oligonucleotides), providing a template for ligation of the adjacent P oligonucleotides. Although not illustrated, ligation of the N group oligonucleotides also may proceed in similar manner to assemble adjacent N oligonucleotides that are annealed to their complementary P oligonucleotide. Assembly of the predetermined nucleic acid fragment may be obtained through ligation of all of the oligonucleotides to generate a double stranded product. However, in some embodiments, a single stranded product of either the positive or negative strand may be obtained. In certain embodiments, a plurality of oligonucleotides may be designed to generate only single-stranded reaction products in a ligation reaction. For example, a first group of oligonucleotides (of either Group P or Group N) may be provided to cover the entire sequence on one strand of the predetermined nucleic acid fragment (on either the positive or negative strand). In contrast, a second group of oligonucleotides (from the complementary group to the first group) may be designed to be long enough to anneal to complementary regions in the first group but not long enough to provide adjacent 5′ and 3′ ends between oligonucleotides in the second group. This provides substrates that are suitable for ligation of oligonucleotides from the first group but not the second group. The result is a single-stranded product having a sequence corresponding to the oligonucleotides in the first group. Again, as with other assembly reactions described herein, a ligase reaction mixture that contains an assembled predetermined nucleic acid fragment also may contain a distribution of smaller fragments resulting from the assembly of a subset of the oligonucleotides.

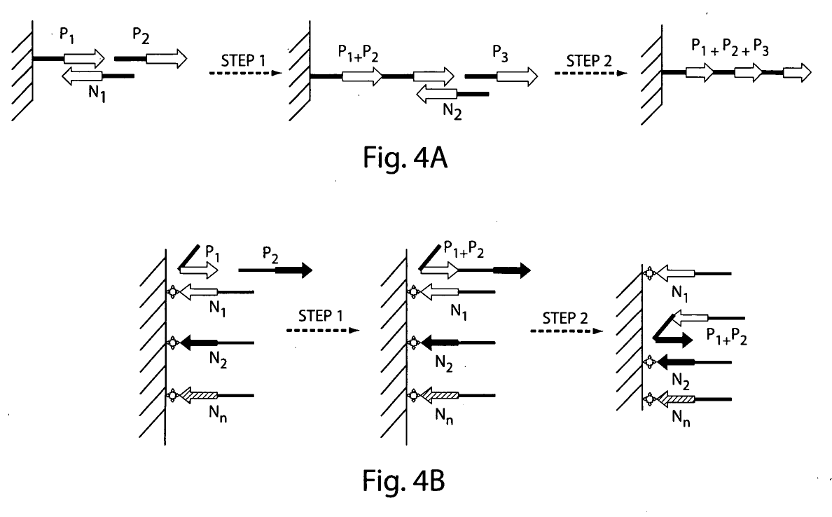

FIG. 4 shows an embodiment of a ligase-based assembly where one or more of the plurality of oligonucleotides is bound to a support. In FIG. 4A, the 5′ most oligonucleotide of the P group oligonucleotides is bound to a support. Ligation of adjacent oligonucleotides in the 5′ to 3′ direction results in the assembly of a predetermined nucleic acid fragment. FIG. 4A illustrates an example where adjacent oligonucleotides P₂ and P₃ are added sequentially. However, the ligation of any two adjacent oligonucleotides from Group P may occur independently and in any order in a ligation reaction mixture. For example, when P₁ is ligated to the 5′ end of N₂, N₂ may be in the form of a single oligonucleotide or it already may be ligated to one or more downstream oligonucleotides (N₃, N₄, etc.). It should be appreciated that for a ligation assembly bound to a support, either the 5′-most (e.g., P₁ for Group P, or N_{T} for Group N) or the 3′-most (e.g., P_{T} for Group P₁ or N₁ for Group N) oligonucleotide may be bound to a support since the reaction can proceed in any direction. In some embodiments, a predetermined nucleic acid fragment may be assembled with a central oligonucleotide (i.e., neither the 5′-most or the 3′-most) that is bound to a support provided that the attachment to the support does not interfere with ligation.

FIG. 4B illustrates an example where a plurality of N group oligonucleotides are bound to a support and a predetermined nucleic acid fragment is assembled from P group oligonucleotides that anneal to their complementary support-bound N group oligonucleotides. Again, FIG. 4B illustrates a sequential addition. However, adjacent P group oligonucleotides may be ligated in any order. Also, the bound oligonucleotides may be attached at their 5′ end, 3′ end, or at any other position provided that the attachment does not interfere with their ability to bind to complementary 5′ and 3′ regions on the oligonucleotides that are being assembled. This reaction may involve one or more reaction condition changes (e.g., temperature cycles) so that ligated oligonucleotides bound to one immobilized N group oligonucleotide can be dissociated from the support and bind to a different immobilized N group oligonucleotide to provide a substrate for ligation to another P group oligonucleotide.

As with other assembly reactions described herein, support-bound ligase reactions (e.g., those illustrated in FIG. 4B) that generate a full length predetermined nucleic acid fragment also may generate a distribution of smaller fragments resulting from the assembly of subsets of the oligonucleotides. A support used in any of the assembly reactions described herein (e.g., polymerase-based, ligase-based, or other assembly reaction) may include any suitable support medium. A support may be solid, porous, a matrix, a gel, beads, beads in a gel, etc. A support may be of any suitable size. A solid support may be provided in any suitable configuration or shape (e.g., a chip, a bead, a gel, a microfluidic channel, a planar surface, a spherical shape, a column, etc.).

As illustrated herein, different oligonucleotide assembly reactions may be used to assemble a plurality of overlapping oligonucleotides (with overlaps that are either 5′/5′, 3′/3′, 5′/3′, complementary, non-complementary, or a combination thereof). Many of these reactions include at least one pair of oligonucleotides (the pair including one oligonucleotide from a first group or P group of oligonucleotides and one oligonucleotide from a second group or N group of oligonucleotides) have overlapping complementary 3′ regions. However, in some embodiments, a predetermined nucleic acid may be assembled from non-overlapping oligonucleotides using blunt-ended ligation reactions. In some embodiments, the order of assembly of the non-overlapping oligonucleotides may be biased by selective phosphorylation of different 5′ ends. In some embodiments, size purification may be used to select for the correct order of assembly. In some embodiments, the correct order of assembly may be promoted by sequentially adding appropriate oligonucleotide substrates into the reaction (e.g., the ligation reaction).

In order to obtain a full-length nucleic acid fragment from a multiplex oligonucleotide assembly reaction, a purification step may be used to remove starting oligonucleotides and/or incompletely assembled fragments. In some embodiments, a purification step may involve chromatography, electrophoresis, or other physical size separation technique. In certain embodiments, a purification step may involve amplifying the full length product. For example, a pair of amplification primers (e.g., PCR primers) that correspond to the predetermined 5′ and 3′ ends of the nucleic acid fragment being assembled will preferentially amplify full length product in an exponential fashion. It should be appreciated that smaller assembled products may be amplified if they contain the predetermined 5′ and 3′ ends. However, such smaller-than-expected products containing the predetermined 5′ and 3′ ends should only be generated if an error occurred during assembly (e.g., resulting in the deletion or omission of one or more regions of the target nucleic acid) and may be removed by size fractionation of the amplified product. Accordingly, a preparation containing a relatively high amount of full length product may be obtained directly by amplifying the product of an assembly reaction using primers that correspond to the predetermined 5′ and 3′ ends. In some embodiments, additional purification (e.g., size selection) techniques may be used to obtain a more purified preparation of amplified full-length nucleic acid fragment.

When designing a plurality of oligonucleotides to assemble a predetermined nucleic acid fragment, the sequence of the predetermined fragment will be provided by the oligonucleotides as described herein. However, the oligonucleotides may contain additional sequence information that may be removed during assembly or may be provided to assist in subsequent manipulations of the assembled nucleic acid fragment. Examples of additional sequences include, but are not limited to, primer recognition sequences for amplification (e.g., PCR primer recognition sequences), restriction enzyme recognition sequences, recombination sequences, other binding or recognition sequences, labeled sequences, etc. In some embodiments, one or more of the 5′-most oligonucleotides, one or more of the 3′-most oligonucleotides, or any combination thereof, may contain one or more additional sequences. In some embodiments, the additional sequence information may be contained in two or more adjacent oligonucleotides on either strand of the predetermined nucleic acid sequence. Accordingly, an assembled nucleic acid fragment may contain additional sequences that may be used to connect the assembled fragment to one or more additional nucleic acid fragments (e.g., one or more other assembled fragments, fragments obtained from other sources, vectors, etc.) via ligation, recombination, polymerase-mediated assembly, etc. In some embodiments, purification may involve cloning one or more assembled nucleic acid fragments. The cloned product may be screened (e.g., sequenced, analyzed for an insert of the expected size, etc.).

In some embodiments, a nucleic acid fragment assembled from a plurality of oligonucleotides may be combined with one or more additional nucleic acid fragments using a polymerase-based and/or a ligase-based extension reaction similar to those described herein for oligonucleotide assembly. Accordingly, one or more overlapping nucleic acid fragments may be combined and assembled to produce a larger nucleic acid fragment as described herein. In certain embodiments, double-stranded overlapping oligonucleotide fragments may be combined. However, single-stranded fragments, or combinations of single-stranded and double-stranded fragments may be combined as described herein. A nucleic acid fragment assembled from a plurality of oligonucleotides may be of any length depending on the number and length of the oligonucleotides used in the assembly reaction. For example, a nucleic acid fragment (either single-stranded or double-stranded) assembled from a plurality of oligonucleotides may be between 50 and 1,000 nucleotides long (for example, about 70 nucleotides long, between 100 and 500 nucleotides long, between 200 and 400 nucleotides long, about 200 nucleotides long, about 300 nucleotides long, about 400 nucleotides long, etc.). One or more such nucleic acid fragments (e.g., with overlapping 3′ and/or 5′ ends) may be assembled to form a larger nucleic acid fragment (single-stranded or double-stranded) as described herein.

A full length product assembled from smaller nucleic acid fragments also may be isolated or purified as described herein (e.g., using a size selection, cloning, selective binding or other suitable purification procedure). In addition, any assembled nucleic acid fragment (e.g., full-length nucleic acid fragment) described herein may be amplified (prior to, as part of, or after, a purification procedure) using appropriate 5′ and 3′ amplification primers.

### Synthetic Oligonucleotides

It should be appreciated that the terms P Group and N Group oligonucleotides are used herein for clarity purposes only, and to illustrate several embodiments of multiplex oligonucleotide assembly. The Group P and Group N oligonucleotides described herein are interchangeable, and may be referred to as first and second groups of oligonucleotides corresponding to sequences on complementary strands of a target nucleic acid fragment.

Oligonucleotides may be synthesized using any suitable technique. For example, oligonucleotides may be synthesized on a column or other support (e.g., a chip). Examples of chip-based synthesis techniques include techniques used in synthesis devices or methods available from Combimatrix, Agilent, Affymetrix, or other sources. A synthetic oligonucleotide may be of any suitable size, for example between 10 and 1,000 nucleotides long (e.g., between 10 and 200, 200 and 500, 500 and 1,000 nucleotides long, or any combination thereof). An assembly reaction may include a plurality of oligonucleotides, each of which independently may be between 10 and 200 nucleotides in length (e.g., between 20 and 150, between 30 and 100, 30 to 90, 30-80, 30-70, 30-60, 35-55, 40-50, or any intermediate number of nucleotides). However, one or more shorter or longer oligonucleotides may be used in certain embodiments.

Oligonucleotides may be provided as single stranded synthetic products. However, in some embodiments, oligonucleotides may be provided as double-stranded preparations including an annealed complementary strand. Oligonucleotides may be molecules of DNA, RNA, PNA, or any combination thereof. A double-stranded oligonucleotide may be produced by amplifying a single-stranded synthetic oligonucleotide or other suitable template (e.g., a sequence in a nucleic acid preparation such as a nucleic acid vector or genomic nucleic acid). Accordingly, a plurality of oligonucleotides designed to have the sequence features described herein may be provided as a plurality of single-stranded oligonucleotides having those feature, or also may be provided along with complementary oligonucleotides. In some embodiments, an oligonucleotide may be phosphorylated (e.g., with a 5′ phosphate). In some embodiments, an oligonucleotide may be non-phosphorylated.

In some embodiments, an oligonucleotide may be amplified using an appropriate primer pair with one primer corresponding to each end of the oligonucleotide (e.g., one that is complementary to the 3′ end of the oligonucleotide and one that is identical to the 5′ end of the oligonucleotide). In some embodiments, an oligonucleotide may be designed to contain a central assembly sequence (designed to be incorporated into the target nucleic acid) flanked by a 5′ amplification sequence (e.g., a 5′ universal sequence) and a 3′ amplification sequence (e.g., a 3′ universal sequence). Amplification primers (e.g., between 10 and 50 nucleotides long, between 15 and 45 nucleotides long, about 25 nucleotides long, etc.) corresponding to the flanking amplification sequences may be used to amplify the oligonucleotide (e.g., one primer may be complementary to the 3′ amplification sequence and one primer may have the same sequence as the 5′ amplification sequence). The amplification sequences then may be removed from the amplified oligonucleotide using any suitable technique to produce an oligonucleotide that contains only the assembly sequence.

In some embodiments, a plurality of different oligonucleotides (e.g., about 5, 10, 50, 100, or more) with different central assembly sequences may have identical 5′ amplification sequences and identical 3′ amplification sequences. These oligonucleotides can all be amplified in the same reaction using the same amplification primers.

A preparation of an oligonucleotide designed to have a certain sequence may include oligonucleotide molecules having the designed sequence in addition to oligonucleotide molecules that contain errors (e.g., that differ from the designed sequence at least at one position). A sequence error may include one or more nucleotide deletions, additions, substitutions (e.g., transversion or transition), inversions, duplications, or any combination of two or more thereof. Oligonucleotide errors may be generated during oligonucleotide synthesis. Different synthetic techniques may be prone to different error profiles and frequencies. In some embodiments, error rates may vary from 1/10 to 1/200 errors per base depending on the synthesis protocol that is used. However, in some embodiments lower error rates may be achieved. Also, the types of errors may depend on the synthetic techniques that are used. For example, in some embodiments chip-based oligonucleotide synthesis may result in relatively more deletions than column-based synthetic techniques.

In some embodiments, one or more oligonucleotide preparations may be processed to remove (or reduce the frequency of) error-containing oligonucleotides. In some embodiments, a hybridization technique may be used wherein an oligonucleotide preparation is hybridized under stringent conditions one or more times to an immobilized oligonucleotide preparation designed to have a complementary sequence. Oligonucleotides that do not bind may be removed in order to selectively or specifically remove oligonucleotides that contain errors that would destabilize hybridization under the conditions used. It should be appreciated that this processing may not remove all error-containing oligonucleotides since many have only one or two sequence errors and may still bind to the immobilized oligonucleotides with sufficient affinity for a fraction of them to remain bound through this selection processing procedure.

In some embodiments, a nucleic acid binding protein or recombinase (e.g., RecA) may be included in one or more of the oligonucleotide processing steps to improve the selection of error free oligonucleotides. For example, by preferentially promoting the hybridization of oligonucleotides that are completely complementary with the immobilized oligonucleotides, the amount of error containing oligonucleotides that are bound may be reduced. As a result, this oligonucleotide processing procedure may remove more error-containing oligonucleotides and generate an oligonucleotide preparation that has a lower error frequency (e.g., with an error rate of less than 1/50, less than 1/100, less than 1/200, less than 1/300, less than 1/400, less than 1/500, less than 1/1,000, or less than 1/2,000 errors per base.

A plurality of oligonucleotides used in an assembly reaction may contain preparations of synthetic oligonucleotides, single-stranded oligonucleotides, double-stranded oligonucleotides, amplification products, oligonucleotides that are processed to remove (or reduce the frequency of) error-containing variants, etc., or any combination of two or more thereof.

In some aspects, a synthetic oligonucleotide may be amplified prior to use. Either strand of a double-stranded amplification product may be used as an assembly oligonucleotide and added to an assembly reaction as described herein. A synthetic oligonucleotide may be amplified using a pair of amplification primers (e.g., a first primer that hybridizes to the 3′ region of the oligonucleotide and a second primer that hybridizes to the 3′ region of the complement of the oligonucleotide). The oligonucleotide may be synthesized on a support such as a chip (e.g., using an ink-jet-based synthesis technology). In some embodiments, the oligonucleotide may be amplified while it is still attached to the support. In some embodiments, the oligonucleotide may be removed or cleaved from the support prior to amplification. The two strands of a double-stranded amplification product may be separated and isolated using any suitable technique. In some embodiments, the two strands may be differentially labeled (e.g., using one or more different molecular weight, affinity, fluorescent, electrostatic, magnetic, and/or other suitable tags). The different labels may be used to purify and/or isolate one or both strands. In some embodiments, biotin may be used as a purification tag. In some embodiments, the strand that is to be used for assembly may be directly purified (e.g., using an affinity or other suitable tag). In some embodiments, the complementary strand is removed (e.g., using an affinity or other suitable tag) and the remaining strand is used for assembly.

In some embodiments, a synthetic oligonucleotide may include a central assembly sequence flanked by 5′ and 3′ amplification sequences. The central assembly sequence is designed for incorporation into an assembled nucleic acid. The flanking sequences are designed for amplification and are not intended to be incorporated into the assembled nucleic acid. The flanking amplification sequences may be used as universal primer sequences to amplify a plurality of different assembly oligonucleotides that share the same amplification sequences but have different central assembly sequences. In some embodiments, the flanking sequences are removed after amplification to produce an oligonucleotide that contains only the assembly sequence.

In some embodiments, one of the two amplification primers may be biotinylated. The nucleic acid strand that incorporates this biotinylated primer during amplification can be affinity purified using streptavidin (e.g., bound to a bead, column, or other surface). In some embodiments, the amplification primers also may be designed to include certain sequence features that can be used to remove the primer regions after amplification in order to produce a single-stranded assembly oligonucleotide that includes the assembly sequence without the flanking amplification sequences.

In some embodiments, the non-biotinylated strand may be used for assembly. The assembly oligonucleotide may be purified by removing the biotinylated complementary strand. In some embodiments, the amplification sequences may be removed if the non-biotinylated primer includes a dU at its 3′ end, and if the amplification sequence recognized by (i.e., complementary to) the biotinylated primer includes at most three of the four nucleotides and the fourth nucleotide is present in the assembly sequence at (or adjacent to) the junction between the amplification sequence and the assembly sequence. After amplification, the double-stranded product is incubated with T4 DNA polymerase (or other polymerase having a suitable editing activity) in the presence of the fourth nucleotide (without any of the nucleotides that are present in the amplification sequence recognized by the biotinylated primer) under appropriate reaction conditions. Under these conditions, the 3′ nucleotides are progressively removed through to the nucleotide that is not present in the amplification sequence (referred to as the fourth nucleotide above). As a result, the amplification sequence that is recognized by the biotinylated primer is removed. The biotinylated strand is then removed. The remaining non-biotinylated strand is then treated with uracil-DNA glycosylase (UDG) to remove the non-biotinylated primer sequence. This technique generates a single-stranded assembly oligonucleotide without the flanking amplification sequences. It should be appreciated that this technique may be used to process a single amplified oligonucleotide preparation or a plurality of different amplified oligonucleotides in a single reaction if they share the same amplification sequence features described above.

In some embodiments, the biotinylated strand may be used for assembly. The assembly oligonucleotide may be obtained directly by isolating the biotinylated strand. In some embodiments, the amplification sequences may be removed if the biotinylated primer includes a dU at its 3′ end, and if the amplification sequence recognized by (i.e., complementary to) the non-biotinylated primer includes at most three of the four nucleotides and the fourth nucleotide is present in the assembly sequence at (or adjacent to) the junction between the amplification sequence and the assembly sequence. After amplification, the double-stranded product is incubated with T4 DNA polymerase (or other polymerase having a suitable editing activity) in the presence of the fourth nucleotide (without any of the nucleotides that are present in the amplification sequence recognized by the non-biotinylated primer) under appropriate reaction conditions. Under these conditions, the 3′ nucleotides are progressively removed through to the nucleotide that is not present in the amplification sequence (referred to as the fourth nucleotide above). As a result, the amplification sequence that is recognized by the non-biotinylated primer is removed. The biotinylated strand is then isolated (and the non-biotinylated strand is removed). The isolated biotinylated strand is then treated with UDG to remove the biotinylated primer sequence. This technique generates a single-stranded assembly oligonucleotide without the flanking amplification sequences. It should be appreciated that this technique may be used to process a single amplified oligonucleotide preparation or a plurality of different amplified oligonucleotides in a single reaction if they share the same amplification sequence features described above.

It should be appreciated that the biotinylated primer may be designed to anneal to either the synthetic oligonucleotide or to its complement for the amplification and purification reactions described above. Similarly, the non-biotinylated primer may be designed to anneal to either strand provided it anneals to the strand that is complementary to the strand recognized by the biotinylated primer.

In certain embodiments, it may be helpful to include one or more modified oligonucleotides in an assembly reaction. An oligonucleotide may be modified by incorporating a modified-base (e.g., a nucleotide analog) during synthesis, by modifying the oligonucleotide after synthesis, or any combination thereof. Examples of modifications include, but are not limited to, one or more of the following: universal bases such as nitroindoles, dP and dK, inosine, uracil; halogenated bases such as BrdU; fluorescent labeled bases; non-radioactive labels such as biotin (as a derivative of dT) and digoxigenin (DIG); 2,4-Dinitrophenyl (DNP); radioactive nucleotides; post-coupling modification such as dR-NH₂ (deoxyribose-NH₂); Acridine (6-chloro-2-methoxiacridine); and spacer phosphoramides which are used during synthesis to add a spacer ‘arm’ into the sequence, such as C3, C8 (octanediol), C9, C12, HEG (hexaethlene glycol) and C18.

It should be appreciated that one or more nucleic acid binding proteins or recombinases are preferably not included in a post-assembly fidelity optimization technique (e.g., a screening technique using a MutS or MutS homolog), because the optimization procedure involves removing error-containing nucleic acids via the production and removal of heteroduplexes. Accordingly, any nucleic acid binding proteins or recombinases (e.g., RecA) that were included in the assembly steps are preferably removed (e.g., by inactivation, column purification or other suitable technique) after assembly and prior to fidelity optimization.

Aspects of the invention may be useful for a range of applications involving the production and/or use of synthetic nucleic acids. As described herein, the invention provides methods for assembling synthetic nucleic acids with increased efficiency. The resulting assembled nucleic acids may be amplified in vitro (e.g., using PCR, LCR, or any suitable amplification technique), amplified in vivo (e.g., via cloning into a suitable vector), isolated and/or purified. An assembled nucleic acid (alone or cloned into a vector) may be transformed into a host cell (e.g., a prokaryotic, eukaryotic, insect, mammalian, or other host cell). In some embodiments, the host cell may be used to propagate the nucleic acid. In certain embodiments, the nucleic acid may be integrated into the genome of the host cell. In some embodiments, the nucleic acid may replace a corresponding nucleic acid region on the genome of the cell (e.g., via homologous recombination). Accordingly, nucleic acids may be used to produce recombinant organisms. In some embodiments, a target nucleic acid may be an entire genome or large fragments of a genome that are used to replace all or part of the genome of a host organism. Recombinant organisms also may be used for a variety of research, industrial, agricultural, and/or medical applications.

Many of the techniques described herein can be used together. For example, concerted assembly may be used to assemble oligonucleotide duplexes and nucleic acid fragments of less than 100 to more than 10,000 base pairs in length (e.g., 100 mers to 500 mers, 500 mers to 1,000 mers, 1,000 mers to 5,000 mers, 5,000 mers to 10,000 mers, 25,000 mers, 50,000 mers, 75,000 mers, 100,000 mers, etc.). In an exemplary embodiment, methods described herein may be used during the assembly of an entire genome (or a large fragment thereof, e.g., about 10%, 20%, 30%, 40%, 50%, 60%, 70%, 80%, 90%, or more) of an organism (e.g., of a viral, bacterial, yeast, or other prokaryotic or eukaryotic organism), optionally incorporating specific modifications into the sequence at one or more desired locations.

Any of the nucleic acid products (e.g., including nucleic acids that are amplified, cloned, purified, isolated, etc.) may be packaged in any suitable format (e.g., in a stable buffer, lyophilized, etc.) for storage and/or shipping (e.g., for shipping to a distribution center or to a customer). Similarly, any of the host cells (e.g., cells transformed with a vector or having a modified genome) may be prepared in a suitable buffer for storage and or transport (e.g., for distribution to a customer). In some embodiments, cells may be frozen. However, other stable cell preparations also may be used.

Host cells may be grown and expanded in culture. Host cells may be used for expressing one or more RNAs or polypeptides of interest (e.g., therapeutic, industrial, agricultural, and/or medical proteins). The expressed polypeptides may be natural polypeptides or non-natural polypeptides. The polypeptides may be isolated or purified for subsequent use.

Accordingly, nucleic acid molecules generated using methods of the invention can be incorporated into a vector. The vector may be a cloning vector or an expression vector. A vector may comprise an origin of replication and one or more selectable markers (e.g., antibiotic resistant markers, auxotrophic markers, etc.). In some embodiments, the vector may be a viral vector. A viral vector may comprise nucleic acid sequences capable of infecting target cells. Similarly, in some embodiments, a prokaryotic expression vector operably linked to an appropriate promoter system can be used to transform target cells. In other embodiments, a eukaryotic vector operably linked to an appropriate promoter system can be used to transfect target cells or tissues.

Transcription and/or translation of the constructs described herein may be carried out in vitro (i.e., using cell-free systems) or in vivo (i.e., expressed in cells). In some embodiments, cell lysates may be prepared. In certain embodiments, expressed RNAs or polypeptides may be isolated or purified. Nucleic acids of the invention also may be used to add detection and/or purification tags to expressed polypeptides or fragments thereof. Examples of polypeptide-based fusion/tag include, but are not limited to, hexa-histidine (His⁶) Myc and HA, and other polypeptides with utility, such as GFP, GST, MBP, chitin and the like. In some embodiments, polypeptides may comprise one or more unnatural amino acid residue(s).

In some embodiments, antibodies can be made against polypeptides or fragment(s) thereof encoded by one or more synthetic nucleic acids.

In certain embodiments, synthetic nucleic acids may be provided as libraries for screening in research and development (e.g., to identify potential therapeutic proteins or peptides, to identify potential protein targets for drug development, etc.)

In some embodiments, a synthetic nucleic acid may be used as a therapeutic (e.g., for gene therapy, or for gene regulation). For example, a synthetic nucleic acid may be administered to a patient in an amount sufficient to express a therapeutic amount of a protein. In other embodiments, a synthetic nucleic acid may be administered to a patient in an amount sufficient to regulate (e.g., down-regulate) the expression of a gene.

It should be appreciated that different acts or embodiments described herein may be performed independently and may be performed at different locations in the United States or outside the United States. For example, each of the acts of receiving an order for a target nucleic acid, analyzing a target nucleic acid sequence, identifying an assembly strategy, designing one or more starting nucleic acids (e.g., oligonucleotides), synthesizing starting nucleic acid(s), purifying starting nucleic acid(s), assembling starting nucleic acid(s), isolating assembled nucleic acid(s), confirming the sequence of assembled nucleic acid(s), manipulating assembled nucleic acid(s) (e.g., amplifying, cloning, inserting into a host genome, etc.), and any other acts or any parts of these acts may be performed independently either at one location or at different sites within the United States or outside the United States. In some embodiments, an assembly procedure may involve a combination of acts that are performed at one site (in the United States or outside the United States) and acts that are performed at one or more remote sites (within the United States or outside the United States).

Aspects of the invention may include automating one or more acts described herein. For example, a sequence analysis may be automated in order to generate a synthesis strategy automatically. The synthesis strategy may include i) the design of the starting nucleic acids that are to be assembled into the target nucleic acid, ii) the choice of the assembly technique(s) to be used, iii) the number of rounds of assembly and error screening or sequencing steps to include, and/or decisions relating to subsequent processing of an assembled target nucleic acid. Similarly, one or more steps of an assembly reaction may be automated using one or more automated sample handling devices (e.g., one or more automated liquid or fluid handling devices). For example, the synthesis and optional selection of starting nucleic acids (e.g., oligonucleotides) may be automated using a nucleic acid synthesizer and automated procedures. Automated devices and procedures may be used to mix reaction reagents, including one or more of the following: starting nucleic acids, buffers, enzymes (e.g., one or more ligases and/or polymerases), nucleotides, nucleic acid binding proteins or recombinases, salts, and any other suitable agents such as stabilizing agents. Automated devices and procedures also may be used to control the reaction conditions. For example, an automated thermal cycler may be used to control reaction temperatures and any temperature cycles that may be used. In some embodiments, a thermal cycler may be automated to provide one or more reaction temperatures or temperature cycles suitable for incubating nucleic acid fragments prior to transformation. Similarly, subsequent purification and analysis of assembled nucleic acid products may be automated. For example, fidelity optimization steps (e.g., a MutS error screening procedure) may be automated using appropriate sample processing devices and associated protocols. Sequencing also may be automated using a sequencing device and automated sequencing protocols. Additional steps (e.g., amplification, cloning, etc.) also may be automated using one or more appropriate devices and related protocols. It should be appreciated that one or more of the device or device components described herein may be combined in a system (e.g., a robotic system). Assembly reaction mixtures (e.g., liquid reaction samples) may be transferred from one component of the system to another using automated devices and procedures (e.g., robotic manipulation and/or transfer of samples and/or sample containers, including automated pipetting devices, etc.). The system and any components thereof may be controlled by a control system.

Accordingly, acts of the invention may be automated using, for example, a computer system (e.g., a computer controlled system). A computer system on which aspects of the invention can be implemented may include a computer for any type of processing (e.g., sequence analysis and/or automated device control as described herein). However, it should be appreciated that certain processing steps may be provided by one or more of the automated devices that are part of the assembly system. In some embodiments, a computer system may include two or more computers. For example, one computer may be coupled, via a network, to a second computer. One computer may perform sequence analysis. The second computer may control one or more of the automated synthesis and assembly devices in the system. In other aspects, additional computers may be included in the network to control one or more of the analysis or processing acts. Each computer may include a memory and processor. The computers can take any form, as the aspects of the present invention are not limited to being implemented on any particular computer platform. Similarly, the network can take any form, including a private network or a public network (e.g., the Internet). Display devices can be associated with one or more of the devices and computers. Alternatively, or in addition, a display device may be located at a remote site and connected for displaying the output of an analysis in accordance with the invention. Connections between the different components of the system may be via wire, wireless transmission, satellite transmission, any other suitable transmission, or any combination of two or more of the above.

In accordance with one embodiment of the present invention for use on a computer system it is contemplated that sequence information (e.g., a target sequence, a processed analysis of the target sequence, etc.) can be obtained and then sent over a public network, such as the Internet, to a remote location to be processed by computer to produce any of the various types of outputs discussed herein (e.g., in connection with oligonucleotide design). However, it should be appreciated that the aspects of the present invention described herein are not limited in that respect, and that numerous other configurations are possible. For example, all of the analysis and processing described herein can alternatively be implemented on a computer that is attached locally to a device, an assembly system, or one or more components of an assembly system. As a further alternative, as opposed to transmitting sequence information (e.g., a target sequence, a processed analysis of the target sequence, etc.) over a communication medium (e.g., the network), the information can be loaded onto a computer readable medium that can then be physically transported to another computer for processing in the manners described herein. In another embodiment, a combination of two or more transmission/delivery techniques may be used. It also should be appreciated that computer implementable programs for performing a sequence analysis or controlling one or more of the devices, systems, or system components described herein also may be transmitted via a network or loaded onto a computer readable medium as described herein. Accordingly, aspects of the invention may involve performing one or more steps within the United States and additional steps outside the United States. In some embodiments, sequence information (e.g., a customer order) may be received at one location (e.g., in one country) and sent to a remote location for processing (e.g., in the same country or in a different country), for example, for sequence analysis to determine a synthesis strategy and/or design oligonucleotides. In certain embodiments, a portion of the sequence analysis may be performed at one site (e.g., in one country) and another portion at another site (e.g., in the same country or in another country). In some embodiments, different steps in the sequence analysis may be performed at multiple sites (e.g., all in one country or in several different countries). The results of a sequence analysis then may be sent to a further site for synthesis. However, in some embodiments, different synthesis and quality control steps may be performed at more than one site (e.g., within one county or in two or more countries). An assembled nucleic acid then may be shipped to a further site (e.g., either to a central shipping center or directly to a client).

Each of the different aspects, embodiments, or acts of the present invention described herein can be independently automated and implemented in any of numerous ways. For example, each aspect, embodiment, or act can be independently implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the present invention comprises at least one computer-readable medium (e.g., a computer memory, a floppy disk, a compact disk, a tape, etc.) encoded with a computer program (i.e., a plurality of instructions), which, when executed on a processor, performs one or more of the above-discussed functions of the present invention. The computer-readable medium can be transportable such that the program stored thereon can be loaded onto any computer system resource to implement one or more functions of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

It should be appreciated that in accordance with several embodiments of the present invention wherein processes are implemented in a computer readable medium, the computer implemented processes may, during the course of their execution, receive input manually (e.g., from a user).

Accordingly, overall system-level control of the assembly devices or components described herein may be performed by a system controller which may provide control signals to the associated nucleic acid synthesizers, liquid handling devices, thermal cyclers, sequencing devices, associated robotic components, as well as other suitable systems for performing the desired input/output or other control functions. Thus, the system controller along with any device controllers together form a controller that controls the operation of a nucleic acid assembly system. The controller may include a general purpose data processing system, which can be a general purpose computer, or network of general purpose computers, and other associated devices, including communications devices, modems, and/or other circuitry or components necessary to perform the desired input/output or other functions. The controller can also be implemented, at least in part, as a single special purpose integrated circuit (e.g., ASIC) or an array of ASICs, each having a main or central processor section for overall, system-level control, and separate sections dedicated to performing various different specific computations, functions and other processes under the control of the central processor section. The controller can also be implemented using a plurality of separate dedicated programmable integrated or other electronic circuits or devices, e.g., hard wired electronic or logic circuits such as discrete element circuits or programmable logic devices. The controller can also include any other components or devices, such as user input/output devices (monitors, displays, printers, a keyboard, a user pointing device, touch screen, or other user interface, etc.), data storage devices, drive motors, linkages, valve controllers, robotic devices, vacuum and other pumps, pressure sensors, detectors, power supplies, pulse sources, communication devices or other electronic circuitry or components, and so on. The controller also may control operation of other portions of a system, such as automated client order processing, quality control, packaging, shipping, billing, etc., to perform other suitable functions known in the art but not described in detail herein.

Aspects of the invention may be useful to streamline nucleic acid assembly reactions. Accordingly, aspects of the invention relate to marketing methods, compositions, kits, devices, and systems for increasing nucleic acid assembly throughput particularly as it relates to the production of cells having altered function as described herein.

Aspects of the invention may be useful for reducing the time and/or cost of production, commercialization, and/or development of synthetic nucleic acids, and/or related compositions. Accordingly, aspects of the invention relate to business methods that involve collaboratively (e.g., with a partner) or independently marketing one or more methods, kits, compositions, devices, or systems for analyzing and/or assembling synthetic nucleic acids as described herein. For example, certain embodiments of the invention may involve marketing a procedure and/or associated devices or systems involving nucleic acid assembly techniques described herein. In some embodiments, synthetic nucleic acids, libraries of synthetic nucleic acids, host cells containing synthetic nucleic acids, expressed polypeptides or proteins, etc., also may be marketed.

Marketing may involve providing information and/or samples relating to methods, kits, compositions, devices, and/or systems described herein. Potential customers or partners may be, for example, companies in the pharmaceutical, biotechnology and agricultural industries, as well as academic centers and government research organizations or institutes. Business applications also may involve generating revenue through sales and/or licenses of methods, kits, compositions, devices, and/or systems of the invention.

### EXAMPLES

### Example 1

### Nucleic Acid Fragment Assembly

Gene assembly via a 2-step PCR method: In step (1), a primerless assembly of oligonucleotides is performed and in step (2) an assembled nucleic acid fragment is amplified in a primer-based amplification.

A 993 base long promoter>EGFP construct was assembled from 50-mer abutting oligonucleotides using a 2-step PCR assembly.

Mixed oligonucleotide pools were prepared as follows: 36 overlapping 50-mer oligonucleotides and two 5′ terminal 59-mers were separated into 4 pools, each corresponding to overlapping 200-300 nucleotide segments of the final construct. The total oligonucleotide concentration in each pool was 5 μM.

A primerless PCR extension reaction was used to stitch (assemble) overlapping oligonucleotides in each pool. The PCR extension reaction mixture was as follows:

| | |
|---|---|
| oligonucleotide pool (5 μM total) | 1.0 μl (~25 nM final each) |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

Assembly was achieved by cycling this mixture through several rounds of denaturing, annealing, and extension reactions as follows:
start 2 min. 95° C.
30 cycles of 95° C. 30 sec., 65° C. 30 sec., 72° C. 1 min.
final 72° C. 2 min. extension step

The resulting product was exposed to amplification conditions to amplify the desired nucleic acid fragments (sub-segments of 200-300 nucleotides). The following PCR mix was used:

| | |
|---|---|
| primerless PCR product | 1.0 μ1 |
| primer 5′ (1.2 μM) | 5 μl (300 nM final) |
| primer 3′ (1.2 μM) | 5 μl (300 nM final) |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

The following PCR cycle conditions were used:
start 2 min. 95° C.
35 cycles of 95° C. 30 sec., 65° C. 30 sec., 72° C. 1 min.
final 72° C. 2 min. extension step

The amplified sub-segments were assembled using another round of primerless PCR as follows. A diluted amplification product was prepared for each sub-segment by diluting each amplified sub-segment PCR product 1:10 (4 μl mix+36 μl dH₂O). This diluted mix was used as follows:

| | |
|---|---|
| diluted sub-segment mix | 1.0 μl |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

The following PCR cycle conditions were used:
start 2 min. 95° C.
30 cycles of 95° C. 30 sec., 65° C. 30 sec., 72° C. 1 min.
final 72° C. 2 min. extension step

The full-length 993 nucleotide long promoter>EGFP was amplified in the following PCR mix:

| | |
|---|---|
| assembled sub-segments | 1.0 μl |
| primer 5′ (1.2 μM) | 5 μl (300 nM final) |
| primer 3′ (1.2 μM) | 5 μl (300 nM final) |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

The following PCR cycle conditions were used:
start 2 min. 95° C.
35 cycles of 95° C. 30 sec., 65° C. 30 sec., 72° C. 1 min.
final 72° C. 2 min. extension step

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications, patents and sequence database entries mentioned herein, including those items listed below, are hereby incorporated by reference in their entirety as if each individual publication or patent was specifically and individually indicated to be incorporated by reference. In addition, the disclosures of co-pending provisional applications Ser. No. 60/801,842, filed May 19, 2006, and Ser. No. 60/801,834, filed May 19, 2006, and the utility and PCT applications claiming priority thereto. In case of conflict, the present application, including any definitions herein, will control.

What is claimed is:

1. A method of altering a cell function comprising
introducing into a cell a nucleic acid comprising a set of genetic elements having recombination sites situated therebetween,
rearranging the genetic elements by recombination at the recombination sites, and
screening the cell for an altered cell function.

2. The method of claim 1, wherein the cell has been modified to delete genomic recombination sites.

3. The method of claim 2, wherein the genomic recombination sites are reduced by 50%.

4. The method of claim 2, wherein the genomic recombination sites are reduced by 90%.

5. The method of claim 1, wherein the cell is a bacterial cell.

6. The method of claim 5, wherein the bacterial cell is an E. coli cell.

7. The method of claim 1, wherein the genetic elements are coding sequences.

8. The method of claim 1, wherein the genetic elements are regulatory and coding sequences.

9. The method of claim 1, wherein the genetic elements are introns and exons.

10. The method of claim 1, further comprising isolating the cell having an altered cell function.

11. The method of claim 1, wherein the nucleic acid is a vector.

12. The method of claim 11, wherein the vector comprises a selection sequence.

13. The method of claim 1, wherein the nucleic acid is integrated into the genome of the cell.

14. The method of claim 1, wherein the recombination sites are identical.

15. The method of claim 1, wherein the recombination sites comprise at least two different types of recombination sites.

16. The method of claim 1, wherein the recombination sites are restriction enzyme sites.

17. The method of claim 1, wherein the recombination sites are homologous recombination sites.

18. The method of claim 1, wherein the recombination sites are susceptible to single or double stranded cuts.

19. A method of producing a cell having an altered cell function comprising
introducing into a cell a nucleic acid comprising a set of genetic elements having recombination sites situated therebetween,
rearranging the genetic elements by allowing recombination between recombination sites, and
isolating a cell having an altered cell function.

20. The method of claim 19, further comprising propagating the cell having an altered function.

21. A method for producing a recombined nucleic acid molecule comprising
producing a cell according to claim 19, and
harvesting from the cell a recombined nucleic acid.
